(19) United States
(12) Patent Application Publication
(10) Pub. No.: US 2004/0115175 A1
(43) Pub. Date: Jun. 17, 2004

(54) Methods for treating disorders of neuronal deficiency with bone marrow-derived cells

(75) Inventors: Helen M. Blau, Menlo Park, CA (US); Timothy Brazelton, Cupertino, CA (US); James M. Weimann, Palo Alto, CA (US)

Correspondence Address: ROPES & GRAY LLP, ONE INTERNATIONAL PLACE, BOSTON, MA, 02110-2624, US

(73) Assignee: The Board of Trustees of the Leland, Palo Alto, CA

(21) Appl. No.: 10/688,747

(22) Filed: Oct. 16, 2003

Related U.S. Application Data
(63) Continuation-in-part of application No. 09/993,045, filed on Nov. 13, 2001.
(60) Provisional application No. 60/247,128, filed on Nov. 10, 2000.

(51) Int. Cl. A61K 45/00

(52) U.S. Cl. 424/93.7

(57) ABSTRACT

The invention provides, among other things, novel methods of treating neurological disorders which result in the loss of neurons (neuronal deficiencies). Bone marrow-derived cells are administered to individuals suffering from neuronal deficiencies. Administration of bone marrow-derived cells results in formation of bone marrow derived neurons, whether formed de novo or as a result of fusion with an existing neuron, thereby replacing or repairing lost or damaged neurons. The methods of the invention may also be used for memory augmentation in memory impaired individuals.

### CROSS-REFERENCE TO RELATED APPLICATIONS

This application is a continuation-in-part of U.S. patent application Ser. No. 09/993,045, filed Nov. 13, 2001, which claims the benefit of U.S. Provisional Application No. 60/247,128, filed Nov. 10, 2000 entitled “Methods for Treating Disorders of Neuronal Deficiency with Bone Marrow-Derived Cells”. The entire contents of both applications are incorporated herein by reference.

### FUNDING

Work described herein was funded, in part, by grant no. AG20961 from the National Institutes of Health. The United States government has certain rights in the invention.

### TECHNICAL FIELD OF THE INVENTION

The invention relates generally to the treatment of neurological disorders, and more particularly to the treatment of neurological conditions characterized by a loss of neurons, damaged neurons, or neurons with inadequate or suboptimal function in the peripheral and/or central nervous system.

### BACKGROUND OF THE INVENTION

Adult bone marrow contains hematopoietic stem cells (HSC) which are capable of restoring the entire range of hematopoietic cells. Thus, bone marrow transplant (BMT) has been used extensively to rescue subjects with bone marrow failure due to myelotoxic chemotherapy/radiotherapy or congenital and/or genetic defects.

While the capacity of HSC to form all hematopoietic cells has been long known, it has been more recently discovered that stem cells present in adult bone marrow also give rise to additional cell types. Donor marrow-derived cells have been found in a variety of tissues. For example, donor marrow-derived liver oval cells have been identified (Peterson et al., 1999, Science, 284:1168-1170), and donor marrow-derived nuclei have been found integrated into skeletal muscle fibers (Gussoni et al., 1999, Nature 401:390-394). Donor marrow-derived cells expressing microglial and astrocytic markers have also been found in the brain following BMT (Eglitis et al., 1997, Proc. Natl. Acad. Sci. USA 94(8):4080-4085; Kennedy et al., 1997, Blood 90(3):986-993)

BMT has also been investigated as a treatment for a number of conditions that do not involve bone marrow failure. For example, enzyme supplementation in lysosomal storage disorders has been attempted by BMT (Krivit et al., 1991, Neuromuscular Disorders 1(6):449-454). Additionally, an ongoing trial in multiple sclerosis seeks to eliminate the autoimmune component by replacing the patient's immune system by allogeneic BMT.

A wide variety of neurological conditions are characterized by a loss of neurons, damaged neurons, or neurons with inadequate or suboptimal function. Some disorders, such as Parkinson's disease, involve loss of a particular type of neuron (dopaminergic neurons), while other disorders, such as stroke, involve the loss of neurons at a particular location (e.g., in an area of the brain supplied by an artery which is blocked during the stroke).

Some attempts have been made to replenish cells lost in such disorders. Implantation of fetal neurons has been attempted as a treatment for Parkinson's disease, and fetal cells have also been used to bridge spinal cord transections. However, cells suitable for such implantation are in extremely limited supply and, because of their fetal origin, ethical questions surround their harvest and use. Additionally, the administration of such cells (which must be directly administered into the brain) causes damage to the brain.

Accordingly, there is a need in the art for new methods for treating disorders involving loss of neurons.

### SUMMARY OF THE INVENTION

In certain aspects, the invention provides new methods for the treatment of neurological conditions characterized by loss of neurons, damaged neurons, or neurons with inadequate or suboptimal function in the central and/or peripheral nervous system (“neuronal deficiencies”). This also includes conditions that may be treated by inducing neuronal loss followed by neuronal replacement. The inventors have found that bone marrow-derived cells are capable of entering the nervous system and forming bone marrow derived neurons. While, in certain embodiments, the precise mechanism by which bone marrow derived neurons form is of secondary importance to the eventual clinical effect, examples of such mechanisms include fusion between a bone marrow derived cell and a neuron (particularly a Purkinje cell) to generate a heterokaryon or transdifferentiation of a bone marrow derived cell to form a neuron. Accordingly, the invention provides methods of treating “neuronal deficiencies” associated with loss of neurons by administering bone marrow-derived cells and as well as the delivery of genetically engineered neuronal progenitors that may be used to treat “neuronal deficiencies.”

The invention provides methods for treating neuronal deficiencies by administering bone marrow-derived cells to an individual having a neuronal deficiency, thereby inducing formation of bone marrow derived neurons in the nervous system of the subject; and ameliorating at least one symptom of the neuronal deficiency. In certain embodiments, the neuronal deficiency arises from a disorder selected from the group consisting of abnormalities of the central autonomic systems, congenital disorders and disorders arising from teratogen exposure, demyelinating diseases, diseases of peripheral nerves, disorders of the hypothalamus and pituitary, disorders of movement, disorders of the spinal cord and vertebral column, epilepsy, hypoxia, increased intracranial pressure, infectious disease, neoplasia, neurodegenerative disorders, neuronal disorders associated with aging and senile dementia, nutritional disorders, perinatal neuropathologies, radiation damage, schizophrenia, single gene disorders, toxic disorders, trauma, vascular disease, and psychiatric disorders other than schizophrenia. The neuronal deficiencies treated by the invention exclude neuronal deficiencies arising from a disorder selected from the group consisting of a lysosomal or peroxisomal disorder, Zellweger's disease, human immunodeficiency virus (HIV) infection, multiple sclerosis (MS), leucodystrophies, adrenomyeloneuropathy, a metachromatic leucodystrophy (including globoid cell leucodystrophy, metachromatic leucodystrophies, and Sanfilipo's disease), sulphatide lipidosis, amyotrophic lateral sclerosis, amyotrophic lateral sclerosis with frontal lobe dementia, a bone marrow ablation treatment, lymphoma, metastases of tumors which do not arise in the nervous system, infantile acid maltase deficiency (Pompe's disease), ceroid lipofuscinosis, a deficiency of GM2 gangliosidase, systemic lupus erythematosus, thrombophilia associated with antiphospholipid antibodies or polycythemia, and anemia including sickle cell disease, beta-thalassemia major, and other thalassemias.

The invention also provides methods for improving memory function in an individual with deficient memory function, by administering bone marrow-derived cells to an individual having deficient memory function, thereby inducing formation of bone marrow derived neurons in the nervous system of the subject; and improving at least one memory function in the individual.

Preferably, the bone marrow-derived cells are autologous, syngeneic, or allogeneic, and the bone-marrow derived cells may be genetically modified. The use of xenogeneic cells is contemplated, but xenogeneic cells are less preferred. The cells may be administered by any method, such as by vascular administration (e.g., intravenously), intrathecally, or locally.

In some embodiments, the bone marrow-derived cells are administered in conjunction with a neuronal factor such as nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene (implicated in X-linked Kallman's syndrome), insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF-1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-.beta.1, -.beta.2, -.beta.3, -.beta.4, and -.beta.5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies).

Also provided are methods for treating a neuron deficiency by administering a bone marrow cell mobilization therapy to an individual having a neuron deficiency, thereby inducing formation of bone marrow derived neurons in the nervous system of the subject; and ameliorating at least one symptom of the neuron deficiency.

Further embodiments of the invention provide for treating a neuron deficiency or for improving memory function by administering bone marrow-derived cells in combination with a bone marrow cell mobilization therapy to an individual having a neuron or memory deficiency, thereby inducing formation of bone marrow derived neurons in the nervous system of the subject; and improving symptoms of the neuronal deficiency or improving memory function in the individual.

### DESCRIPTION OF THE DRAWINGS

FIG. 1. Controls. Identification of Purkinje neurons and specificity of the human X and Y chromosome DNA probes. Sections from control female and male cerebella were processed with a mixture of the X (red) and Y (green) probes. The nucleus was counterstained with To-Pro-3 (blue) and imaged by using a scanning confocal microscope at 1-μm optical sections. (A-C) Labeling in a female control cerebellum. (A) Two Purkinje neurons are clearly defined with a large nucleus surrounded by a large cytoplasmic region. Each cell has one X chromosome labeled (red arrows). (B and C) Enlargements of the Purkinje neurons in A without the blue nuclear label to facilitate visualization of the chromosome. Note that many of the granular neurons have two X chromosomes in contrast to the large Purkinje cells. (D-F) Male control cerebellum with three Purkinje neurons labeled. Note that one neuron has one X chromosome (red arrow), another has an X and a Y chromosome (red and green arrows), whereas the third has only one Y chromosome (green arrow). (E and F) Enlargements of D without the blue nuclear label. The white arrowhead in D and F shows a Y chromosome from a closely abutting cell. Note that many of the granular neurons contain one X (red) and one Y (green) chromosome. (Scale bars: 20 μm, A and D; 10 μm, B, C, E, and F.)

FIG. 2. Male donor-derived cells in the circulation as well as the parenchyma of the cerebellum. (A) Donor-derived male blood cells are present in blood vessels in female recipients (field is representative of 20 examples imaged). (B) Same image as in A with the blue nuclear labeling removed. (C) Occasionally donor-derived cells can be found in the granular and molecular layers of the cerebellum possibly en route to the Purkinje layer (field is representative of >30 images captured). (D) Same image as in C with the blue nuclear labeling removed. (Scale bar: 10 μm.)

FIG. 3. Evidence of male Y chromosome in Purkinje neurons. (A-C) Three examples of male bone marrow-derived nuclei in Purkinje cell. Each neuron has one X (red arrow) and one Y (green arrow) chromosome. These Purkinje neurons appear to be well integrated into the surrounding cerebellum with a mature morphology including dendrites. (D-F) Same images as A-C with the blue nuclear counterstain removed to highlight the red and green probes. The single X chromosome imaged in B and E has a dumbbell shape, a phenomenon observed infrequently. This chromosome is enlarged in the Inset (E) to demonstrate that it is a single chromosome. Note the wisp of red-labeled chromatin connecting the two lobes that are 1.18 μm apart, whereas the Y chromosome is 3.52 μm from the X. (Scale bar: 20 μm.)

FIG. 4. Evidence for fusion between donor-derived bone marrow cells and host Purkinje neurons. Two examples of triple sex chromosomes in Purkinje neurons. (A) Male to female transplant. There are two X (red) and one Y (green) chromosomes in this cell. (B) Same image as A without the nuclear counterstain. The two X chromosomes are 4.22 μm apart, whereas the Y chromosome is 4.44 μm and 6.08 μm separated from the two X chromosomes. The distance between each chromosome indicates that these are each unique chromosomes. (C) Male to female transplant. There are three distinct X chromosomes (red arrows) in this cell. These chromosomes are 4.16 μm, 6.12 μm, and 4.61 μm apart. (Scale bar: 10 μm.)

FIG. 5: GFP-positive Purkinje neurons in the cerebellum. (a) A schematic representation of a mouse brain, showing the anterior olfactory bulb (Of), cerebral cortex (Ctx), thalamus (Th) and the caudally located cerebellum (Cb). (b). In thick sections (45 μm) cut from the cerebellum of a post-bone-marrow-transplanted mouse, individual donor-derived GFP-positive Purkinje neurons are evident in the Purkinje cell layer (PCL). The dendrites from these cells extend into the cell-sparse molecular layer (ML), whereas their axon projects through the granular cell layer (GCL) and is the only output connection from the cerebellum to the rest of the brain. Three lobes of the cerebellum in the box in a can be seen in b. Note the many bone-marrow-derived (GFP-positive) cells in the parenchyma. (c) A high-power laser-scanning confocal image of this cell shows its many synaptic spines and single output axon (arrow). The two GFP-positive BMDC cells are probably microglia or macrophages in PCL and ML (arrowheads). Scale bars represent 2 mm in a, 100 μm in b and 50 μm in c.

FIG. 6: Marker expression in GFP-positive Purkinje neurons. Immunohistochemistry with specific antibodies demonstrates the presence of Purkinje-specific markers, but not marrow markers. (a, b) Calbindin, a calcium-binding protein, stains the dendrites and somata of Purkinje cells. All of the GFP-positive Purkinje neurons were also calbindin-positive (arrow; 20/20 cells), whereas none of the microglia or other cell types in the cerebellum were calbindin-positive. (c, d) GFP-positive Purkinje cells were negative for the pan-haematopoietic marker CD45 (arrow; 0/15 cells), whereas donor and host microglia were positive (insets). (e-h) These GFP-positive Purkinje cells were also negative for the macrophage/microglia markers CD11b (arrow; 0/8 cells) and F4/80 (arrow; 0/12 cells). Note that other BMDCs remain positive for these markers (arrowheads). (i,j). The microglia calcium-binding protein Iba1 did not stain any of the GFP-positive neuronal soma or dendrites (0/7 cells; arrow) but strongly labelled adjacent microglia (arrowheads). Scale bar represents 50 μm in all panels.

FIG. 7: Time course of GFP-positive Purkinje neuron appearance. (a) Mice were bone-marrow-transplanted at two months of age and cerebella were collected and analysed at various times. The number of GFP-positive Purkinje cells increased in a linear manner over time, with a linear regression of 0.92. One animal analysed at 18 months had a higher than expected number of cells (n=60) that may reflect some aspect of the ageing process. This one data point was not included in the graph. (b, c) All of the GFP-positive Purkinje cells observed contained two nuclei. This Purkinje cell has a distinctive dendritic tree with many synaptic spines and an axon exiting the soma at the left. One of the two nuclei in the cell is compact (arrowhead) and is the putative BMD nucleus. The other nucleus has dispersed chromatin similar to other Purkinje neurons (arrows). In 752 control Purkinje neurons from transplanted and normal mice, no binucleated cells were observed. Scale bar represents 20 μm.

FIG. 8: Fusion of a male BMDC to a female Purkinje neuron. (a) A Texas-Red-labelled Y chromosome probe was used to detect donor-derived male nuclei by FISH. A single 1-μm confocal optical section through a 12-μm section of a GFP-positive Purkinje cell containing two nuclei. (b) After protease digestion and hybridization, it is evident that the top nucleus (arrowhead) has a Y chromosome and is donor-derived. Note that some displacement of the nuclei occurs through digestion and in situ processing. (c) Another double-nuclei GFP-positive cell. The host nucleus in d is beneath the male donor-derived nucleus shown in c (arrowhead). The host nucleus visible in d does not possess a Y chromosome. Scale bar represents 20 μm.

FIG. 9: Changes in nuclear morphology within heterokaryons over time. In some cases one of the two nuclei has compact chromatin (a, e), in others the chromatin is beginning to look dispersed (b, f), whereas in others the two nuclei appear identical (c, g). In some cases, both appear as normal Purkinje nuclei with highly dispersed chromatin and a prominent nucleolus (d, h). Arrowheads indicate donor-derived bone marrow nucleus and arrows point to Purkinje-like dispersed nuclei. (i) The nuclear appearance of both nuclei in the fused cells was measured. Nuclei that were 20% the diameter of normal Purkinje nuclei were considered compact, whereas nuclei that were 60% or greater in diameter were considered as dispersed. The ratio of dispersed-to-compact nuclei per animal is plotted over a period of 16 months post-transplant. Scale bar represents 20 μm in panels a-h.

FIG. 10: Flow cytometry of bone marrow from the L7-GFP mouse. FACS analysis, showing that bone marrow cells do not express the Purkinje neuron-specific transgene, L7-pcp2-GFP. Bone marrow was dissociated from two wild-type mice, two GFP transgenic mice and four L7-GFP transgenic mice. Comparison of the FACS profiles shows that the wild-type and L7-GFP mice display no fluorescence when compared with the GFP bone marrow. Gating of PI-labelled cells (dead cells) excluded them from the contour profiles.

FIG. 11: Evidence for reprogramming of BMDCs after fusion to Purkinje neurons. (a) Low-power image of L7-GFP bone-marrow-derived Purkinje neuron with the soma in the PCL and a dendrite extending into the ML. (bd) High-power images of three L7-GFP bone-marrow-derived Purkinje neurons. All eight of the L7-GFP neurons had double nuclei. In all images, Green represents GFP and Red represents To-Pro3. Scale bar represents 50 μm in a and 20 μm in b-d.

### DESCRIPTION OF THE INVENTION

In part, the invention relates to the discovery that administration of cells contained in the bone marrow results in formation of bone marrow derived neurons in the central nervous system. These cells do not express astrocytic, microglial, nor hematopoietic markers. Formation of neurons by bone marrow-derived cells has not, to the inventors' knowledge, been previously described.

The invention provides methods of treating neurological indications which involve the loss of neurons, damaged neurons, or neurons with inadequate or suboptimal function in the central and/or peripheral nervous system (“neuron deficiencies”) by administering bone marrow-derived cells. Administration of bone marrow-derived cells results in improvement in one or more symptoms of the neuron deficiencies being treated. Bone marrow-derived cells are already extensively used in clinical practice, although for other indications, and provide the advantage of being readily accessible (e.g., compared to fetal brain cells).

Additionally, the invention provides methods for increasing central and/or peripheral nervous system neurons or changing their function and/or connections (i.e., plasticity) by administering bone marrow-derived cells. These methods are useful for improving and/or stabilizing mental function, such as memory, particularly short term memory function, for correcting dysfunctions such as epilepsy, ataxias, and psychological disorders such as disorders of mood and/or affect, or for delivering genes to the CNS.

Also provided are methods for treating symptoms of a neuronal deficiency by administering a bone marrow cell mobilization therapy to a subject having a neuronal deficiency. Mobilization of bone marrow cells results in the formation of bone marrow derived neurons in the nervous system of the subject and further results in improvement in one or more symptoms of the neuronal deficiencies being treated.

Definitions

As used herein, the term “subject” or “individual” refers to a vertebrate, and includes avians and mammals. The term “mammal” refers to any individual of a mammalian species, and includes large animals (cows, sheep, horses and the like), sport animals (including dogs and cats), and primates (including old world monkeys, new world monkeys, apes, humans, and the like).

As used herein, the term “treating” refers to ameliorating, improving, reducing, or stabilizing one or more symptoms of a disorder or undesired condition, as well as slowing progression of one or more symptoms of a neuronal deficiency.

The term “neuronal deficiency”, as used herein, refers to a neurological disorder characterized by the actual or potential loss of neurons, damaged neurons, or neurons with inadequate or suboptimal function in the central and/or peripheral nervous system. Neuronal deficiencies include neurological disorders arising from disorders including abnormalities of the central autonomic systems, congenital disorders and disorders arising from teratogen exposure, demyelinating diseases, diseases of peripheral nerves, disorders of the hypothalamus and pituitary, disorders of memory or suboptimal memory, disorders of movement, disorders of the spinal cord and vertebral column, epilepsy, hypoxia, increased intracranial pressure, infectious disease, neoplasia, neurodegenerative disorders, neuronal disorders associated with aging and senile dementia, nutritional and metabolic disorders, perinatal neuropathologies, radiation damage, schizophrenia, psychiatric disorders other than schizophrenia, single gene disorders, toxic disorders, trauma, and vascular disease. As used herein, the term “neuronal deficiencies” excludes a number of diseases/disorders/syndromes: lysosomal and peroxisomal disorders, Zellweger's disease, neuronal deficiencies arising from human immunodeficiency virus (HIV) infection, multiple sclerosis (MS), adrenoleucodystrophy, adrenomyeloneuropathy, metachromatic leucodystrophies, sulphatide lipidoses, globoid cell leucodystrophy (Krabbe's disease, galactosylceramide lipidosis), amyotrophic lateral sclerosis, sporadic amyotrophic lateral sclerosis, amyotrophic lateral sclerosis with frontal lobe dementia, familial amyotrophic lateral sclerosis, and familial amyotrophic lateral sclerosis with frontal lobe dementia, bone marrow ablation (e.g., by chemotherapy), lymphomas (e.g., primary malignant lymphomas, secondary lymphomas, and plasma cell tumors) as well as metastases of tumors which do not arise in the nervous system, infantile acid maltase deficiency (Pompe's disease), metachromatic leucodystrophy, ceroid lipofuscinosis, deficiencies of GM2 gangliosidases, Sanfilipo's disease, leucodystrophy, systemic lupus erythematosus, thrombophilia associated with antiphospholipid antibodies or polycythemia, anemias (including sickle cell disease, beta-thalassemia major, and other thalassemias).

Neuronal deficiencies are listed below and organized into groups based on type of disease or location. However, as many disorders have complex outcomes and involve multiple sites and pathologies within the nervous system, it should be noted that the inventors contemplate that the disorders listed below should not be limited to the pathology or location of the group in which they are listed, but include all nervous system locations and pathologies affected by the named disorder.

The term “abnormalities of the central autonomic systems”, as used herein, refers to those neuronal deficiencies involving a loss of neurons, damaged neurons, or neurons with inadequate or suboptimal function due to central autonomic failure, including primary and secondary causes of autonomic failure such as progressive autonomic failure (including progressive autonomic failure with Lewy bodies, with multiple system atrophy, and due to postganglionic pathology), dopamine beta-hydroxylase deficiency, structural lesions of the spinal cord, brain stem, corticolimbic or hypothalamic regions, cerebrovascular disease, botulism, acute autonomic neuropathy, and peripheral neuropathies such as diabetic, amyloid, inflammatory, alcoholic, toxic, drug-related, chronic renal failure, paraneoplastic, connective tissue disease, acute intermittent porphyria, and familial neuropathy. The term “abnormalities of the central autonomic systems” excludes neuropathies related to lymphoreticular proliferative disorders (including lymphoma, leukemia, myeloma, and polycythemia vera).

The term “congenital disorders and teratogens” refers to those neuronal deficiencies involving a loss of neurons, damaged neurons, or neurons with inadequate or suboptimal function associated with spinal cord malformations (including secondary degenerations), syringomyelia, syringobulbia, meningocele, spina bifida occulta, hydromyelia, diplomyelia, diastematomyelia, anomalies of the septum pellucidum (including secondary destructions), neuronal migration defects (e.g., laminar neuronal hetertopias, microdysgenesis, and hippocampal anomalies), encephaloclastic defects, megalencephaly, malformations/hypoplasias/dysplasias/atrophies of the cerebrum and cerebellum, pontoneocerebellar hypoplasia, granular layer aplasia, olivopontocerebellar atrophy in association with carbohydrate deficient glycoprotein (CDG) deficiency (disialotransferrin developmental deficiency syndrome), crossed cerebellar atrophy, cerebellar heterotopias, cerebellar cortical dysplasia, brain stem malformations, olivary dysplasia, dentate dysplasia, dentato-olivary dysplasia, arthogryposis multiplex congenta (AMC) syndrome (it should be noted that the term “AMC”, as used herein, excludes AMC associated with Zellweger's syndrome), malformations involving the nervous system associated with trisomy 21, trisomy 13, trisomy 14, trisomy 18, trisomy 8 mosaicism, fragile X syndrome, Lhermitte-Duclos disease, Dandy-Walker syndrome, Joubert's syndrome, septo-optic dysplasia, Fukuyama congenital muscular dystrophy, Walker-Warburg syndrome, cerebro-ocular dysplasia-muscular dystrophy syndrome (COD-MD), Mobius syndrome, Sturge-Weber syndrome, arachnoid cysts, phakomatosis, tuberous sclerosis, Bourneville's disease, hypomelanosis of Ito, Von Recklinghausen's disease, hydrocephalus, fetal alcohol syndrome, maternal phenylketonuria, maternal diabetes mellitus, and maternal infection with teratogenic infectious agents (such as rubella, cytomegalovirus, Herpes simplex, Herpes zoster, toxoplasmosis, and the like), vascular malformations involving the CNS and/or PNS, and maternal exposure to teratogens such as alcohol, carbamazepine, hyperthermia, methyl mercury, phenyloin, retinoids, valproic acid, varicella, warfarin, and X-irradiation.

The term “demyelinating disease” refers to neuron deficiencies involving a loss of neurons, damaged neurons, or neurons with inadequate function associated with demyelination due to viral causes such as progressive multifocal leucoencephalopathy, subacute sclerosing panencephalitis, canine distemper encephalomyelitis, mouse hepatitis virus (JHM) encephalomyelitis, Theiler's murine virus encephalomyelitis, Semliki Forest virus encephalomyelitis, Visna, Herpes simplex virus type I and type II infections, and Human T lymphotrophic virus Type I (HTLV I) associated myelopathy (tropical spastic paraplegia), phenylketonuria, autoimmune (or suspected autoimmune) causes including perivenous encephalomyelitis (postinfectious encephalomyelitis, postvaccinal encephalomyelitis), rabies postvaccinal encephalomyelitis, acute hemorrhagic leucoencephalitis (Hurst's disease), nutritional/metabolic causes which include disorders such as Marchiafava-Bignami disease, vitamin B12 deficiency (subacute combined degeneration), and central pontine myelinosis, toxic causes including hexachlorophene intoxication, and periventricular leucoencephalopathy associated with combined anti-mitotic medication and radiotherapy, and other causes which include disorders such as prolonged cerebral edema, hypoxic-ischemic leucoencephalopathy (carbon monoxide poisoning, anoxic and ischemic anoxia), and cerebrospinal fluid exchange, perivenous encephalomyelitis which includes acute disseminated encephalomyelitis, postinfectious encephalomyelitis, postvaccinal encephalomyelitis, and acute perivascular myelinoclasis, rabies postvaccinal encephalomyelitis, acute hemorrhagic leucoencephalitis (Hurst's disease), acquired hypomyelinogenesis congenita, starvation, protein deprivation, essential fatty acid deficiency, copper deficiency, vitamin B12 deficiency, electrolyte-induced demyelination, spinal cord compression, cerebrospinal fluid exchange, and X-irradiation of the CNS in young and mature animals. As used herein, a “demyelinating disease due to a viral cause” excludes human immunodeficiency virus (HIV) encephalopathy and HIV vacuolar myelopathy. “Demyelinating disease due to an autoimmune cause” excludes multiple sclerosis (MS), including variants of the disease. The term “demyelinating disease due to a genetic cause” excludes adrenoleucodystrophy, adrenomyeloneuropathy, metachromatic leucodystrophies, sulphatide lipidoses, and globoid cell leucodystrophy (Krabbe's disease, galactosylceramide lipidosis), neuropathies related to lymphoreticular proliferative disorders (including lymphoma, leukemia, myeloma, and polycythemia vera and hereditary neuropathies affecting peripheral nerves exclude metachromatic leucodystrophy (sulphatide lipidosis)), adrenoleucodystrophy, adrenomyeloneuropathy, G M.sub. 1 gangliosidosis, G M.sub.2 gangliosidosis, Gaucher's disease, Niemann-Pick disease (including type A (type I) and type C (type II), Fabry's disease (angiokeratoma corporis diffusum), Wolman's disease, and Batten's disease.

The term “diseases of peripheral nerves”, as used herein, refers to those neuronal deficiencies involving a loss of neurons, damaged neurons, or neurons with inadequate or suboptimal function due to neuronal and/or axonal degeneration/dysfunction including that associated with trauma, crush injury, stretch injury, transection, radiation neuropathy, distal axonopathy, ischemic injury, chronic nerve compression, cold injury, polyglucosan bodies, Strachan's syndrome, alcoholic neuropathy, peripheral neuropathies due to toxic neuropathies (including those due to environmental agents and biological agents such as including acrylamide, buckthorn, carbon disulphide, carbon monoxide, dimethylaminopropionitrile (DMAPN), diptheria toxin (including diphtheritic neuropathy), ethylene oxide, hexacarbons, metals (including arsenic, lead, mercury, and thallium), organophosphorus esters, drug-induced neuropathies (including those associated with colchicine, gold, isoniazid nucleosides (dideoxycytidine (ddC), dideoxyinosine (ddI), and stavudine (d4T)), platinum, taxol, and vincristine), neuropathies related to system metabolic disorders (e.g., uremic neuropathy and those associated with diabetes mellitus, hypoglycemia, and hypothyroidism) and amyloid neuropathies (including those associated with primary amyloidosis). The term “disorders of peripheral nerves” excludes neuropathies associated with HIV infection and neuropathies related to lymphoreticular proliferative disorders (including lymphoma, leukemia, myeloma, and polycythemia vera and hereditary neuropathies affecting peripheral nerves exclude metachromatic leucodystrophy (sulphatide lipidosis), globoid cell leucodystrophy (Krabbe's disease, galactosylceramide lipidosis), Refsum's disease, adrenoleucodystrophy, adrenomyeloneuropathy, G M.sub.1 gangliosidosis, G M.sub.2 gangliosidosis, Gaucher's disease, Niemann-Pick disease (including type A (type I) and type C (type II), Fabry's disease (angiokeratoma corporis diffusum), Farber's disease, Wolman's disease, amyloidosis associated with myeloma Waldenstrom's macroglobulinemia, and Batten's disease.

The term “disorders of the hypothalamus and pituitary”, as used herein, refers to those neuronal deficiencies involving a loss of neurons, damaged neurons, or neurons with inadequate or suboptimal function due to disorders of the hypothalamus and pituitary such as hypothalamic and posterior pituitary hyperfunction, hypothalamic and posterior pituitary hypofunction, malformations and hamartomas of the pituitary and hypothalamus, inflammatory lesions, infectious diseases, metabolic disorders, degenerative diseases, and vascular diseases.

The term “disorders of movement”, as used herein, refers to neuronal deficiencies involving a loss of neurons, damaged neurons, or neurons with inadequate or suboptimal function due to akinetic rigid movement disorders, Parkinsonism including Parkinson's disease, idiopathic Parkinson's disease, drug-induced parkinsonism, vascular pseudoparkinsonism, arteriosclerotic pseudoparkinsonism, Alzheimer-type changes, frontotemporal neurodegenerative disorders, juvenile parkinsonism, toxin-related parkinsonism, Guam parkinsonism, parkinsonism dementia complex of Guam, and postencephalitic parkinsonism, conditions characterized by abnormal stiffness such as stiff man syndrome, progressive encephalomyelitis with rigidity and Isaac's syndrome, hyperkinetic movement disorders including Huntington's disease, metabolic derangements, drug-induced chorea, and focal lesion-induced chorea, myoclonal disorders such as Creutzfeldt-Jakob disease, Lewy body disease, and Alzheimer's disease, dystonias, tic disorders including Gilles de la Tourette syndrome, ataxic disorders including Friedreich's ataxia, ataxia-telengiectasia, autosomal dominant cerebellar ataxias, episodic ataxias, and Wolfram's syndrome, motor neuron disorders including motor neuron disorders secondary to an infectious disease or toxin exposure (e.g., post-polio syndrome, and syphilis infection) and spinal muscular atrophies. The term “movement disorders” also includes disorders affecting basal ganglia including thalamic lesions (e.g., as occurs in Friedreich's ataxia, fatal familial insomnia, and in isolated thalamic degeneration) pallidal degenerations (e.g. as occurs in pure pallidal degeneration, pallidoluysial degeneration, pallidonigral degeneration, and pallidonigroluysial degeneration), neuroaxonal dystrophy and related disorders (including physiological neuroaxonal dystrophy, primary neuroaxonal dystrophies and secondary neuroaxonal dystrophies), disorders associated with mineralization of basal ganglia (including hypoparathyroidism, familial psychosis, pupus cerebritis, and folate deficiency, but excluding carbonic anhydrase II deficiency) disorders associated with calcification of basal ganglia (striatopallidodentate calcification, brain calcinosis, Fahr's disease), striatal necrosis (including that associated with hypoxia, hypoglycemia, carbon monoxide poisoning, and the like) and neuroleptic malignant syndrome (including the hyperthermia and multiorgan failure associated with 3,4-methylenedioxymethamphetamine (MDMA, ‘Ecstasy’). The term “disorders of movement”, particularly “hyperkinetic movement disorders”, excludes Batten's disease and systemic lupus erythematosus. Additionally, the term “disorders of movement”, particularly “motor neuron disorders”, excludes amyotrophic lateral sclerosis, sporadic amyotrophic lateral sclerosis, amyotrophic lateral sclerosis with frontal lobe dementia, familial amyotrophic lateral sclerosis, and familial amyotrophic lateral sclerosis with frontal lobe dementia, and HIV infection.

The term “disorders of the spinal cord and vertebral column,” as used herein, refers to those neuronal deficiencies involving a loss of neurons, damaged neurons, or neurons with inadequate or suboptimal function due to a disorder of the spinal cord and/or vertebral column, including vascular diseases such as occlusive vascular disease: (e.g., resulting in ischemic myelopathy), compression of spinal cord, diseases of the vertebral column affecting the spinal cord including intervertebral disc release and spondylosis, bony abnormalities in the region of the foramen magnum, rheumatoid arthritis and ankylosing spondylitis, spinal cord compression, infectious diseases involving vertebrae and meninges, neoplastic processes involving vertebrae and meninges, trauma including penetrating injuries, non-penetrating injuries, post-traumatic syringomyelia, partial or total spinal cord transection, and chronic adhesive spinal arachnoiditis. The term “disorders of the spinal cord and vertebral column” excludes neuropathies related to lymphoreticular proliferative disorders (including lymphoma, leukemia, myeloma, and polycythemia vera).

The term “epilepsy” refers to those neuronal deficiencies characterized by chronic, recurrent paroxysmal changes in neurological function. Each episode is referred to as a “seizure”, and may present with motor, sensory, autonomic, or psychic symptoms. Seizures with motor symptoms are “convulsive” seizures”. Epilepsy includes status epilepticus, chronic loss of neurons, reactive gliosis, and iatrogenic damage relating to surgical or medical treatment. The term “epilepsy” includes idiopathic epilepsy, primary epilepsies, age-related onset epilepsies, childhood epilepsies, epilepsies secondary to other disorders, such as malformations, infantile Huntington's disease, vascular malformation(s), infection(s) and infectious diseases such as meningitis, encephalitis, parasite infection, and malaria, post-traumatic epilepsy, gliotic scar associated epilepsy, and epilepsies associated with intracranial tumors, infarcts, febrile episodes, and the like. As used herein, the term “epilepsy” excludes epilepsy associated with juvenile Gaucher disease, neuropathies related to lymphoreticular proliferative disorders (including lymphoma, leukemia, myeloma, and polycythemia vera), and Krabbe's disease.

The term “hypoxia”, as used herein, refers to those neuronal deficiencies involving a loss of neurons, damaged neurons, or neurons with inadequate or suboptimal function due to hypoxias including hypoxic hypoxia, anemic hypoxia, stagnant hypoxia (including cardiac arrest encephalopathy and transient global ischemia), non-perfused brain (including respirator brain and permanent global ischemia), and histotoxic hypoxia) (and including hypoxia associated with carbon monoxide poisoning, air embolism, vascular disruption/blockage (including stroke and embolism) and decompression sickness. “Hypoxia” of the central nervous system, and particularly the brain, results in ischemic lesions. In certain embodiments, individual ischemic lesions in the CNS of a subject having a hypoxic neuron deficiency are less than about 5%, 2.5%, or 1% of total brain volume, although individual lesions may “fuse” to form aggregate lesions which are greater than 5%, 2.5%, or 1% of total brain volume (aggregate lesions can be recognized by the shape of their aggregated borders). As used herein, the term “hypoxia” excludes hypoxia related to lymphoreticular proliferative disorders (including lymphoma, leukemia, myeloma, and polycythemia vera).

The term “increased intracranial pressure” refers to those neuronal deficiencies involving a loss of neurons, damaged neurons, or neurons with inadequate or suboptimal function due to raised intracranial pressure. Increased intracranial pressure may be due to a variety of causes, including changes in cerebrospinal fluid production or absorption or intracranial blood volume, brain swelling and edema, intracranial expanding lesions (including hemorrhage, hydrocephalus (including obstructive (non-communicating) hydrocephalus), and benign intracranial hypertension.

The term “infectious disease” refers to those neuronal deficiencies involving a loss of neurons, damaged neurons, or neurons with inadequate or suboptimal function due to an infectious disease such as a viral infection (e.g., herpes virus infection, poliovirus infection (e.g. poliovirus acute encephalomyelitis), arbovirus infection (e.g., acute encephalitis caused by arboviruses), mumps, measles, rubella and the like), parasitic infections such as protozoal infections (e.g., amoebiasis, cerebral malaria, toxoplasmosis, and the like), fungal infections including Aspergillosis, Candidiasis, and the like, bacterial infections including pyogenic infections (e.g., abscess), mycoplasma infections such as Sarcoidosis, and prion diseases including scrapie. The term “infectious disease”, as used herein, excludes HIV infections (AIDS) as well as infections occuring in individuals with HIV infection (e.g., Aspergillosis or Candidiasis).

As used herein, the term “lysosomal storage disorder” refers to an inborn error of metabolism which results in a build up of one or more substances in the lysosomal compartment of cells of an individual afflicted with the disorder. Lysosomal storage disorders may result in mental and/or physical disabilities and may additionally reduce the life expectancy of the afflicted individual, depending on the identity and severity of the particular lysosomal storage disorder. The known lysosomal storage disorders include: Pompe's disease (acid-a1,4-glucosidase deficiency), G.sub.M1,-gangliosidosis including the infantile form (type 1), pseudo-Hurler disease, Tay-Sachs with visceral involvement, familial neurovisceral lipidosis, Landing's disease, generalized gangliosidosis, and adult G.sub.M1-gangliosidosis (type 3), Tay-Sachs disease (.beta.-hexosaminidase A deficiency), G.sub.M2-gangliosidosis including the infantile (Tay-Sachs) forms (types B, O and AB) and the infantile, late infantile, juvenile or adult forms (types B and B1), G.sub.M3-gangliosidosis, G.sub.D3-gangliosidosis, Sandhoff disease (.beta.-hexosaminidase A & B deficiency), Fabry disease (.alpha.-galactosidase A deficiency), Gaucher disease (glucocerebrosidase deficiency) including Type 1 (chronic non-neuronopathic Gaucher's disease), Type 2 (acute neuronopathic Gaucher's disease), Type 3 (the Norrbottnian type, or subacute or juvenile neuronopathic Gaucher's disease), types A and B Nieman-Pick (acid sphingomyelinase deficiency), type C Nieman-Pick (cholesterol esterification defect), type D Nieman-Pick, Farber disease (acid ceramidase deficiency), Wolman's disease (acid lipase deficiency), mucopolysccharidosis (MPS) IH (Hurler's syndrome/disease, .alpha.-L-iduronidase deficiency), MPS IS (Scheie syndrome/disease, .alpha.-L-iduronidase deficiency), MPS IH/S (Hurler-Scheie syndrome/disease, .alpha.-L-iduronidase deficiency), MPS II (Hunter's syndrome/disease, iduronate sulfatase deficiency), MPS II subtype A (severe Hunter's syndrome/disease), MPS II subtype B (mild Hunter's syndrome/disease), MPS III (Sanfilippo's syndrome/disease), MPS III subtype A (subtype A Sanfilippo's syndrome/disease, heparan N-sulfatase deficiency), MPS III subtype B (subtype B Sanfilippo's syndrome/disease, .alpha.-N-acetylglucosaminidase deficiency), MPS III subtype C (subtype C Sanfilippo's syndrome/disease, acetyl-CoA-glucosaminide acetyltransferase deficiency), MPS III subtype D (subtype D Sanfilippo's syndrome/disease, N-acetylglucosamine-6-sulfatase deficiency), MPS IV (Morquio's syndrome/disease), MPS IV subtype A (galactosamine-6-sulfatase deficiency aka Morquio A or severe Morquio's syndrome/disease), MPS IV subtype B (Morquio B/mild Morquio's syndrome/disease, .beta.-galactosidase deficiency), MPS VI (Maroteaux-Lamy's syndrome/disease, arylsulfatase B deficiency), MPS VI subtype A (severe Maroteaux-Lamy's syndrome/disease), MPS VI subtype B (mild Maroteaux-Lamy's syndrome/disease), MPS VII (Sly's syndrome/disease, .beta.-glucuronidase deficiency), mannosidoses including mannosidosis, alpha-mannosidosis, severe infantile alpha-mannosidosis, severe infantile type I alpha-mannosidosis infantile alpha-mannosidosis, type I alpha-mannosidosis, juvenile-adult alpha-mannosidosis, type II alpha-mannosidosis, mild alpha-mannosidosis, juvenile-adult type II alpha-mannosidosis, beta-mannosidosis, and other variants, fucosidosis (.alpha.-L-fucosidase deficiency) including type I fucosidosis, infantile fucosidosis, and type II fucosidosis, aspartylglucosaminuria (N-aspartyl-.beta.-glucosaminidase), sialidosis (.alpha.-neuraminadase deficiency, aka mucolipidosis I), galactosialidosis (lysosomal protective protein deficiency, aka Goldberg syndrome), Schindler disease (.alpha.-N-acetyl-galactosaminidase deficiency), mucolipidosis II (N-acetylglucosamine-1-phosphotransferase deficiency, aka I-cell disease), mucolipidosis III (N-acetylglucosamine-1-phosphotransferase deficiency, aka pseudo-Hurler polydystrophy), cystinosis (cystine transport protein deficiency) including severe neuropathic cystinosis, infantile cystinosis, intermediate cystinosis, childhood cystinosis, juvenile cystinosis, adult cystinosis, and benign cystinosis, sialurias including sialuria, Salla disease (sialic acid transport protein deficiency), and infantile sialic acid storage disease (sialic acid transport protein deficiency), infantile neuronal ceroid lipofuscinosis (palmitoyl-protein thioesterase deficiency), mucolipidosis IV, and prosaposin (saposin A, B, C or D deficiency), G.sub.M1-gangliosidosis including the infantile form (type 1), Batten's disease including neuronal ceroid lipofuscinosis (NCL), Bielschowsky-Jansky disease, Spielmeyer-Vogt-Sjogren disease, Stengel's disease, amaurotic familial idiocy, cerebral lipidosis with onset past infancy, cerebromacular degeneration, diffuse lipofuscinosis, heredofamilial lipidosis, maculocerebral degeneration, neurovisceral storage disease with curvilinear bodies, polyunsaturated fatty acid lipidosis, infantile Batten's disease, (CLN1), Late-infantile Batten's disease (CLN2), Juvenile Batten's disease (CLN3), Adult Batten's disease (CLN4), Kufs' disease, Finnish variant late-infantile Batten's disease (CLN5), early juvenile Batten's disease, Juvenile Batten disease with granular osmiophilic deposits, infantile NCL, late infantile NCL, juvenile NCL, and other a typical variants of Battens disease, Congenital amaurotic idiocy, Neuronal storage associated with osteopetrosis as described by Takahashi et. al, (Pathol Res Pract, 186:697-706, 1990) and Ambler et. al., (Neurology, 33:437-441, 1988), Niemann-Pick disease including the group I variants (including Groups A and B) and the Group II variants (Groups C, D, and the pure visceral form) and also includes juvenile dystonic lipidosis, juvenile dystonic idiocy without amaurosis, a typical cerebral lipidosis, a typical juvenile lipidosis, subacute Niemann-Pick disease, juvenile Niemann-Pick disease, ophthalmoplegic lipidosis, neurovisceral storage disease with vertical supranuclear ophthalmoplegia, Neville-Lake syndrome, Neville's disease, subacute neurovisceral lipidosis, lactosylceramidosis, sea-blue histiocyte disease, syndrome of the sea-blue histiocyte, chronic reticuloendothelial cell storage disease, and Nova Scotian variant of Niemann-Pick disease, leucodystrophies, mucosulphatidoses associated with one or more defects in many different sulphatases including the Austin variant of metachromatic leucodystrophy and multiple sulphatase deficiency, Krabbe's leucodystrophy including Krabbe's globoid cell leucodystrophy, and Johnny McLeod's disease, neuraminidase deficiency including mucolipidosis I, neuraminidase deficiency group A, neuraminidase deficiency group A subtype 1/i (no dysmorphic features), Cherry-red spot myoclonus syndrome, sialidosis type I, Cherry-red spot/myoclonus syndrome, neuraminidase deficiency group A subtype 2/ii (with dysmorphic features: childhood type), lipomucopolysaccharidosis, Goldberg's syndrome, Sialidosis type II, neuraminidase deficiency group A subtype 3/iii (with dysmorphic features: infantile, severe type), neuraminidase deficiency group B (neuraminidase/beta-galactosidase deficiency[galactosidosis]) subtype 1/i (juvenile-adult type with no or mild dysmorphic features), and neuraminidase deficiency group B (neuraminidase/beta-galactosidase deficiency[galactosidosis]) subtype 2/ii (infantile type with severe or mild dysmorphic features), 1 cell disease, pseudo-Hurler polydystrophy, and other disorders involving defects in N-acetylglucosamine-1-phosphotra-nsferase, mucolipidosis IV, Type II glycogenosis, Pompe's disease, generalized glycogenesis, acid maltase deficiency, lysosomal glycogen storage disease, lysosomal glycogen storage disease without acid maltase deficiency, Farber's lipogranulomatosis, acid esterase deficiency, acid lipase deficiency, and cholesteryl ester storage disease, peroxisomal disorders of infancy, disorders of defective peroxisome assembly such as Zellweger's cerebro-hepato-renal syndrome, and dihydroxyacetone-phosphate acyl transferase deficiency, neonatal adrenoleucodystrophy, adrenoleucodystrophy, infantile Refsum's disease, pseudo infantile Refsum's syndrome, hyperpipecolic acidemia, Zellweger-like syndrome, rhizomelic-infantile chondroplasia punctata (classical type), disorders with single enzyme defects including pseudo-Zellweger's syndrome, 3-oxoacyl coenzyme A thiolase dysfunction/deficiency, pseudo-neonatal adrenoleucodystrophy, peroxisomal bifunctional enzyme deficiency, rhizomelic chondroplasia punctata, bifunctional enzyme deficiency, trihydroxycholestanoic acidemia, pipecolic acidemia (isolated), Refsum's disease, a typical Refsum's disease, glutaric aciduria type III, primary hyperoxaluria, acatalasemia, mevalonic aciduria, Conradi-Hunermann syndrome/disease, X-linked chondroplasia punctata, Conradi-Hunermann chondroplasia punctata, Sjogren-Larsson syndrome as well as other disorders with dysfunctions in peroxisomes, Schilder's disease with adrenal insufficiency, classical adrenoleucodystrophy, childhood adrenoleucodystrophy, mild adrenoleucodystrophy, adult adrenoleucodystrophy, adrenomyeloneuropathy (AMN), adolescent adrenoleucodystrophy, adult cerebral adrenoleucodystrophy, Addison only adrenoleucodystrophy, presymptomatic adrenoleucodystrophy, asymptomatic adrenoleucodystrophy, primary hyperoxaluria type I, and alanine:glyoxalate aminotransferase deficiency/dysfunction/mistargetting and other leucodystrophies including Canavan's disease (van Bogaert and Bertrand type of spongy degeneration), infantile Canavan's disease, congenital Canavan's disease, rapidly progressive Canavan's disease, rapidly progressive infantile Canavan's disease, juvenile Canavan's disease, protracted Canavan's disease, Pelizaeus-Merzbacher disease including the variety of genetic defects in myelin proteolipid protein which give rise to the variety of subtypes of Pelizaeus-Merzbacher disease, Alexander's disease, infantile Alexander's disease, childhood Alexander's disease, juvenile Alexander's disease, adult Alexander's disease, and adult onset Alexander's disease. As used herein, the term “leucodystrophies” refers to adrenoleucodystrophy, adrenomyeloleucodystrophy, and metachromatic leucodystrophies (including sulphatide lipidosis, aryl sulphatase deficiency, cerebroside sulphatase deficiency), globoid cell leucodystrophy (including Krabbe's disease, galactosylceramide lipidosis), X-linked adrenoleucodystrophy (Schilder's disease), neonatal adrenoleukodystrophy, mucosulphatidosis (multiple sulphatase deficiency, Austin variant of metachromatic leucodystrophy), and X-linked leucodystrophy).

The term “neoplasia”, as used herein, refers to those neuronal deficiencies involving a loss of neurons, damaged neurons, or neurons with inadequate or suboptimal function due to tumors of the nervous system including tumors of neuroepithelial tissue (e.g., astrocytic and ependymal tumors, mixed gliomas, tumors of the choroid plexus and neuroepithelial tumors of uncertain origin such as astroblastomas, polar spongioblastomas, and gliomatosis cerebri), neuronal and neuronal-glial tumors, tumors of the pineal region, embryonal tumors (e.g., medulloepitheliomas, ependymoblastomas, neuroblastomas), tumors of peripheral nerves such as schwannomas and neurofibromas, tumors of the meninges, mesenchymal non-meningothelial tumors, germ cell tumors and tumor-like conditions such as cysts, and plasma cell granulomas, paraneoplastic syndromes, optic nerve tumors of the hypothalamus, posterior pituitary and sellar region. As used herein, the term “neoplasia” excludes lymphomas (e.g., primary malignant lymphomas, secondary lymphomas, and Plasma cell tumors) leukemias, myelomas and polycythemia vera as well as nervous system metastases of tumors which do not arise in the nervous system. The term “paraneoplastic syndromes” excludes paraneoplastic syndromes associated with lymphomas (e.g., primary malignant lymphomas, secondary lymphomas, and Plasma cell tumors) leukemias, myelomas and polycythemia vera.

The term “neurodegenerative disorders” refers to those neuronal deficiencies involving a loss of neurons, damaged neurons, or neurons with inadequate or suboptimal function associated with neurodegenerative disorders such as autosomal recessive proximal spinal muscular atrophy, primary subcortical degenerations (such as Parkinson's disease, multiple system atrophy, Huntington's disease, and progressive supranuclear palsy), familiar and spontaneous Alzheimer's disease, and prion diseases. The term “neurodegenerative diseases” excludes muscular dystrophies, multiple sclerosis, and acquired immunodeficiency syndrome (AIDS), as well as Pick's disease, infantile acid maltase deficiency (Pompe's disease). The term “metabolic neuropathy neurodegenerative diseases” excludes leukodystrophies such as metachromatic leucodystrophy and globoid leucodystrophy.

The term “neuronal disorder associated with aging and senile dementia” refers to those neuronal deficiencies involving a loss of neurons, damaged neurons, or neurons with inadequate or suboptimal function due to aging or senile dementia such as changes in dendritic trees (e.g., loss of dendritic spines, swellings, varicosities, and distortions of the horizontal branches, progressive swelling of the cell body, loss of basal dendrites, loss of branches of the apical shaft, loss of terminal branches, loss of apical shaft), decreased synaptic density, shrinkage of neurons, increased lipofuscin content, decreased Nissyl substance, decreases in brain volume, periventricular leucaraiosis, leucaraiosis, accumulation of senile plaques (including amyloid plaques, argyrophilic plaques), accumulation of neurotic plaques, accumulation of non-neurotic plaques, and accumulation of neurofibrillary tangles.

The term “nutritional disorders” refers to those neuronal deficiencies involving a loss of neurons, damaged neurons, or neurons with inadequate or suboptimal function associated with nutritional disorders such as chronic protein-calorie malnutrition or malabsorption (e.g., anorexia nervosa, short bowel syndrome as well as malabsorption associated with cystic fibrosis) and vitamin deficiencies (e.g., thiamine, niacin, vitamin B12 or E deficiency). The term “nutritional disorders,” as used herein, excludes lysosomal storage disorders.

The term “perinatal neuropathologies” refers to those neuronal deficiencies involving a loss of neurons, damaged neurons, or neurons with inadequate or suboptimal function which present during the perinatal period, such as neuronal deficiencies associated disorders such as mental retardation, toxic/metabolic damage such as white and/or grey matter lesions resulting from hypoxia/ischemia (e.g., neuronal cell injury and neuronal necrosis), and prenatal exposure to maternal cocaine and the associated vascular-related lesions, neuronal damage resulting from extracorporeal membrane oxygenation (ECMO) and ECMO associated vascular-related lesions, neuronal damage resulting from congenital heart disease and congenital heart disease associated vascular lesions, kemicterus, neuronal damage resulting from cerebral hemorrhage, neuronal damage resulting from infections such as cytomegalovirus (CMV), neonatal meningitis (including organisms such as Group B streptococcus, E. coli, Staphylococcus, Pseudomonas, Klebsiella), infantile meningitis (including organisms such as Haemophilus influenzae, meningococcemia, and pneumococcus), fungal infections (including organisms such as Candida albicans, Mucor, Crytococcus, Coccidioides, and Aspergillus), TORCH infections (including organisms such as Toxoplasma gondii, rubella, cytomegalovirus, varicella zoster, cocksackie A and B, echovirus, poliovirus, Treponemapallidium, and herpes simplex type 1 and 2), trauma, (e.g., birth trauma, subdural hematoma, spinal cord injury), and neuronal deficiencies resulting from sudden infant death syndrome (SIDS), neuronal deficiencies resulting from neoplasia. As used herein, “perinatal neuropathologies”, and particularly storage/metabolic perinatal neuropathologies, excludes Ceroid lipofuscinosis, deficiencies of GM2 gangliosidases, leucodystrophies such as Sanfilipo's disease, and Zellweger's disease. As used herein, “perinatal neuropathologies”, and particularly “TORCH infections resulting in perinatal neuropathologies” excludes infection by human immunodeficiency virus (HIV). In certain embodiments, the term “perinatal neuropathologies” excludes all lysosomal storage disorders.

The term “radiation damage”, as used herein, refers to those neuronal disorders involving a loss of neurons, damaged neurons, or neurons with inadequate or suboptimal function associated with X-irradiation induced damage (including acute, early delayed, and late delayed reactions), radiation induced-lesions of the white matter of the brain, the spinal cord, and/or the peripheral nerves. As used herein, “radiation damage” refers to neuronal deficiencies caused by at least 20 Gy for individuals of more than about 2 years of age, at least about 3 Gy for individuals newborn through 2 years of age, and at least about 1 Gy for individuals prenataly exposed to X-radiation.

The term “schizophrenia, as used herein, refers to a neuronal deficiency due to a schizophrenic disorder, including disorganized schizophrenia (DSM-IV 295.1), hebephrenic schizophrenia, paranoid schizophrenia (DSM-IV 295.3), residual schizophrenia (DSM-IV 295.6), catatonic schizophrenia (DSM-IV 295.2), simple schizophrenia, simple deteriorative disorder, undifferentiated type schizophrenia (DSM-IV 295.9), and schizophrenia associated with specific syndrome complexes including 1) hallucinations and delusions, 2) disorganized behavior including positive formal thought disorder, bizarre behavior and inappropriate affect, 3) primary, enduring or deficit symptoms, including restricted affective experience and expression, diminished drive, and poverty of thought.

As used herein, the term “single gene disorder” refers to a neuronal deficiency due to a defect in a single gene, including Aicardi's syndrome, Angelman's syndrome, Aniridia/Wilm's association, Apert's syndrome, Holoprosencephaly 1, Holoprosencephaly 2, Holoprosencephaly 3, Kallmann's syndrome, Meckel-Gruber syndrome, Miller-Dieker syndrome, Neu-Laxova syndrome, Pallister-Hall syndrome, Pettigrew's syndrome, Prader-Willi syndrome, Sacral agenesis (Currarino triad), Tuberous sclerosis, Waardenburg syndrome type I, Warburg's syndrome, and X-linked hydrocephalus. The term “single gene disorder”, as used herein, excludes lysosomal storage disorders.

The term “toxic disorders”, as used herein, refers to neuronal deficiencies involving a loss of neurons, damaged neurons, or neurons with inadequate or suboptimal function associated with exposure to toxins such as metallic toxins including aluminum, arsenic (organic and inorganic), bismuth, cadmium, lead (inorganic and organic), manganese, mercury (inorganic and organic), alkyl mercury compounds, methyl mercury, platinum, tellurium, thallium, tin, alkyl tin compounds, triethyl tin, trimethyl tin, as well as syndromes associated with metallic intoxications such as chronic aluminum-induced motor neuron degeneration, dialysis encephalopathy, and human manganism, environmental toxins including acrylamide, acrylamide monomer, carbon disulphide, L-tryptophan, alcohol, ethyl alcohol, ethanol, methanol, methyl alcohol, methyl ester, hexacarbon solvents, n-hexane, methyl-n-butyl ketone, 2,5-hexanedione, formaldehyde, MPTP (N-methyl-4-phenyl-1,2,3,6-tetrahydropyridine), MPP+(1-methyl-4-phenylpyridinium), organophosphorus compounds, toluene, styrene, trichloroethylene, xylene, and other solvents, rapeseed oil, oleyl-anilide, as well as syndromes associated with these intoxications such as eosinophilia-myalgia syndrome, fetal alcohol syndrome, “glue sniffing” syndrome, Parkinsonian-like syndrome, organophosphate toxicity syndromes, solvent abuse encephalopathy, and toxic oil syndrome, drug toxicities due to drugs such as Amiodarone, Chloroquine, Clioquinol, Colchicine, Dapsone, Disulfiram (ANTABUSE.RTM.), Hexachlorophene (PhisoHex), Isoniazid, Isonicotinic acid hydrazide, Mevacor (LOVASTATIN.RTM.), Nitroimidazoles (metronidazole, misonidazole), Perhexiline maleate, Phenyloin (DILANTIN.RTM.), Pyridoxine, naturally-occurring (biological) toxic compounds including Buckthorn toxin, Cycad (seeds contain cycasin and beta-N-methylamino-L-alanine), Lathyrus sativus (leads to lathyrism, neurolathyrism), 3-Nitropropionic acid (ingestion of fungus Arthinium), Domoic acid, and Psychosine. The term “toxic disorders”, as used herein, excludes neuronal deficiencies associated with treatment of HIV infection (e.g., treatment with ZIDOVUDINE RTM.) or treatment with methotrexate, vincristine, or paclitaxel (TAXOL RTM.). In some embodiments, the term “toxic disorders” excludes neuronal deficiencies associated with administration of amphotericin B.

The term “trauma” refers to neuronal deficiencies involving a loss of neurons, damaged neurons, or neurons with inadequate or suboptimal function associated with trauma such as blunt (non-missile) trauma and including focal injury (which includes contusions, intracranial hemorrhage, hematoma, subdural hygroma, tissue tear hemorrhages associated with diffuse axonal injury, and intraventricular hemorrhage), traumatic separation, cranial nerve injury, and injury to blood vessels in the CNS and servicing the CNS, and fat embolism(s)), diffuse injury (which includes diffuse axonal injury, hypoxic (ischemic) brain damage (including that associated with infarction, episodes of hypemia, raised ICP, transient failures of cerebral perfusion pressure, hypotension, cardiac arrest status epilepticus and hypoglycemia), diffuse brain swelling (including that either around focal injuries, or in one or both hemispheres), diffuse vascular injury, multiple small hemorrhages (e.g., petechial hemorrhages) (including that associated with hematological complications associated with thrombocytopenia, small blood vessel disease (often due to sepsis) and adverse drug reactions), dementia pugilistica (punch-drunk syndrome), injury resulting in focal or diffuse (multi-focal) brain damage (including adverse outcomes such as severe neurological disabilities, vegetative state, post-traumatic epilepsy, and progressive neurological disease)), missile head injury, and injury associated with neurosurgery, other surgery, or biopsy. As used herein, the term “trauma” excludes hypoxia related to lymphoreticular proliferative disorders (including lymphoma, leukemia, myeloma, and polycythemia vera).

The term “vascular disease”, as used herein, refers to neuronal deficiencies involving a loss of neurons, damaged neurons, or neurons with inadequate or suboptimal function associated with vascular diseases including diseases of blood vessels (including stroke, atherosclerosis, hypertensive angiopathy, inflammatory diseases including non-infectious vasculitides, and infectious vasculitides including bacterial vasculitis, granulomatous, and viral vasculitides, aneurysms, vascular malformations, arterial spasm, vascular dementia, and cerebral amyloid angiopathies.-The term “vascular disease” also includes hematologic disorders which result in blood flow abnormalities (including thrombosis, thrombophilia, hyperviscosity, and platelet abnormalities). As used herein, the term vascular diseases excludes systemic lupus erythematosus, thrombophilia associated with antiphospholipid antibodies or polycythemia, anemias (including Sickle cell disease, beta-Thalassemia major, and other thalassemias), lymphoreticular proliferative disorders (including lymphoma, leukemia, myeloma, and polycythemia vera), and viral vasculitides associated with HIV infection. The term “cerebral amyloid neuropathies” excludes amyloidosis associated with myeloma and Waldenstrom's macroglobulinemia.

The term “psychiatric disorders other than schizophrenia”, as used herein, refers to psychiatric disorders including dementia (DSM-IV 290, 290.1, 290.1, 290.11, 290.12, 290.13, 290.2, 290.21, 290.3, 290.4, 290.41, 290.42, 290.43, 294.1, 294.1, 294.1, 294.1, 294.8, 294.8) alcohol induced disorders (DSM-IV 291.1, 291.2, 291.81 291.9), substance abuse-related psychiatric disorders (DSM-IV 292, 292.11, 292.12, 292.81-0.84, 292.89, 292.9), psychiatric disorders secondary to a medical condition (DSM-IV 293.83, 293.89, 293.9, 294), cognitive disorders (DSM-IV 294.9), depressive disorders (DSM-IV 296.3, 296.31-0.35, 311), bipolar disorders (DSM-IV 296.4, 296.41-0.46, 296.5, 296.51-0.56, 296.6, 296.61-0.66, 296.7, 296.8, 296.89), mood disorders (DSM-IV 296.9), psychotic disorders (DSM-IV 298.9), autistism (DSM-IV 299), narcissistic personality disorder (DSM-IV 301.81), tic disorders (DSM-WV 307.2, 307.22), Tourette's disorder (DSM-IV 307.23), pain disorders (DSM-IV 307.8, 307.89) posttraumatic stress disorder (DSM-IV 309.81), mental retardation, (DSM-IV 317, 318, 318.1, 318.2, 319), neuroleptic-induced Parkinsonism (DMS-IV 332.1), narcolepsy (DSM-IV 347), age-related cognitive decline (DSM-WV 780.9), borderline intellectual functioning (DSM-IV V62.89). The term “psychiatric disorders other than schizophrenia”, as used herein, specifically excludes dementia due to a lysosomal storage disorder (e.g., DSM-IV 290.1) or HIV infection (DSM-IV 294.9).

As used herein, the term “ablative regimen” refers to a treatment protocol or regimen which reduces and/or eliminated circulating white cells, hematopoietic stem cells, and/or hematopoietic precursor cells. Ablative regimens are well known in the art, and generally involve the administration of gamma irradiation and/or cytotoxic chemotherapy.

As used herein, the term “neuronal factors” refers to factors which affect the proliferation, differentiation and/or survival of neurons. Neuronal factors include growth factors, neurotransmitters and the like, as long as they have the biological activity of affecting the proliferation, differentiation and/or survival of neurons.

As used herein, the term “comprising” and its cognates are used in their inclusive sense (i.e., synonymously with “including” and its cognates).

As used herein, the singular form “a”, “an”, and “the” includes plural references unless indicated otherwise. For example, “a” bone marrow-derived cell includes one or more bone marrow-derived cells.

It should be noted that the inventors have disclosed herein a number of disorders involving neuronal deficiencies which have a number of variants and subtypes and which may be referred to by different names by those of skill in the art. The inventors contemplate the inclusion of all subtypes and variants of the neuronal deficiencies disclosed herein, even if the particular subtype or variant is not specifically disclosed. Similarly, this disclosure encompasses all synonyms, eponyms, equivalent terms and/or translations of a particular disorder/syndrome/disease, even if the synonyms, equivalent terms, and/or translations are not specifically disclosed herein. Additional synonyms, eponyms, equivalent terms and translations of neuronal deficiency names, as well as variants and subtypes may be found in GREEFIELD's NEUROPATHOLOGY, (Graham et al., eds., 6th ed., 1997, Oxford University Press, N.Y.) and KAPLAN AND SADOCK's COMPREHENSIVE TEXTBOOK OF PSYCHIATRY (Sadock et al., eds., 7th ed., 2000, Lippincott Williams and Wilkins).

The practice of the present invention will employ, unless otherwise indicated, conventional techniques of molecular biology, microbiology, cell biology, recombinant DNA, and medicine, which are within the skill of the art. See, e.g., Sambrook et al., MOLECULAR CLONING: A LABORATORY MANUAL, 2nd edition (1989); CURRENT PROTOCOLS IN MOLECULAR BIOLOGY, (F. M. Ausubel et al. eds., 1987); the series METHODS IN ENZYMOLOGY (Academic Press, Inc.); PCR 2: A PRACTICAL APPROACH (M. J. McPherson, B. D. Hames and G. R. Taylor eds., 1995); ANIMAL CELL CULTURE (R. I. Freshney. Ed., 1987); and ANTIBODIES: A LABORATORY MANUAL (Harlow et al. eds., 1987).

Bone marrow-derived cells may administered to a subject in need of augmentation (e.g., repair or replacement) of central or peripheral nervous system neurons. The subject, may suffer from a neuronal deficiency, or may otherwise be in need of neuronal augmentation, such as for improvement of memory. The bone marrow-derived cells may be autologous (i.e., derived from the same individual) or syngeneic (i.e., derived from a genetically identical individual, such as a syngeneic littermate or an identical twin), although allogeneic bone marrow-derived cells (i.e., cells derived from a genetically different individual of the same species) are also contemplated. Although less preferred, xenogeneic (i.e., derived from a different species than the recipient) bone marrow-derived cells, such as bone marrow-derived cells from transgenic pigs, may also be administered. When the donor bone marrow-derived cells are xenogeneic, it is preferred that the cells are obtained from an individual of a species within the same order, more preferably the same superfamily or family (e.g., when the recipient is a human, it is preferred that the donor bone marrow-derived cells are derived from a primate, more preferably a member of the superfamily Hominoidea).

Bone marrow-derived cells may be obtained from any stage of development of the donor individual, including prenatal (e.g., embryonic or fetal), infant (e.g., from birth to approximately three years of age in humans), child (e.g., from about three years of age to about 13 years of age in humans), adolescent (e.g., from about 13 years of age to about 18 years of age in humans), young adult (e.g., from about 18 years of age to about 35 years of age in humans), adult (from about 35 years of age to about 55 years of age in humans) or elderly (e.g. from about 55 years and beyond of age in humans).

In some embodiments, the bone marrow-derived cells are administered as unfractionated bone marrow. It is preferred, however, particularly for allogeneic or xenogeneic transplants that the bone marrow be fractionated to enrich for the bone marrow-derived cells prior to administration. Methods of fractionation are well known in the art, and generally involve both positive selection (i.e., retention of cells based on a particular property) and negative selection (i.e., elimination of cells based on a particular property). As will be apparent to one of skill in the art, the particular properties (e.g., surface markers) that are used for positive and negative selection will depend on the species of the donor bone marrow-derived cells.

Methods for fractionation and enrichment of bone marrow-derived cells are best characterized for human and mouse cells, but those of ordinary skill in the art can select homologous markers and methods for fractionating and enriching bone marrow-derived cells from other species.

When the donor bone marrow-derived cells are human, there are a variety of methods for fractionating bone marrow and enriching bone marrow-derived cells known in the art. Positive selection methods such as enriching for cells expressing CD34, and Thy-1 may be used, and negative selection methods which remove or reduce cells expressing CD3, CD10, CD11b, CD14, CD16, CD15, CD16, CD19, CD20, CD32, CD45, CD45R/B220, Ly6G, TER-119 may also be used alone or in combination with positive selection techniques. When the donor bone marrow-derived cells are not autologous, it is preferred that negative selection be performed on the cell preparation to reduce or eliminate differentiated T cells, thereby reducing the risk of graft versus host disease (GVHD).

Generally, methods used for selection/enrichment of bone marrow-derived cells will utilize immunoaffinity technology, although density centrifugation methods are also useful. Immunoaffinity technology may take a variety of forms, as is well known in the art, but generally utilizes an antibody or antibody derivative in combination with some type of segregation technology. The segregation technology generally results in physical segregation of cells bound by the antibody and cells not bound by the antibody, although in some instances the segregation technology which kills the cells bound by the antibody may be used for negative selection.

Any suitable immunoaffinity technology may be utilized for selection/enrichment of bone marrow-derived cells, including fluorescence-activated cell sorting (FACS), panning, immunomagnetic separation, immunoaffinity chromatography, antibody-mediated complement fixation, immunotoxin, density gradient segregation, and the like. After processing in the immunoaffinity process, the desired cells (the cells bound by the immunoaffinity reagent in the case of positive selection, and cells not bound by the immunoaffinity reagent in the case of negative selection) are collected and either subjected to further rounds of immunoaffinity selection/enrichment, or reserved for administration to the patient.

Immununoaffinity selection/enrichment is typically carried out by incubating a preparation of cells comprising bone marrow-derived cells with an antibody or antibody-derived affinity reagent (e.g., an antibody specific for a given surface marker), then utilizing the bound affinity reagent to select either for or against the cells to which the antibody is bound. The selection process generally involves a physical separation, such as can be accomplished by directing droplets containing single cells into different containers depending on the presence or absence of bound affinity reagent (FACS), by utilizing an antibody bound (directly or indirectly) to a solid phase substrate (panning, immunoaffinity chromatography), or by utilizing a magnetic field to collect the cells which are bound to magnetic particles via the affinity reagent (immunomagnetic separation). Alternately, undesirable cells may be eliminated from the bone marrow-derived cell preparation using an affinity reagent which directs a cytotoxic insult to the cells bound by the affinity reagent. The cytotoxic insult may be activated by the affinity reagent (e.g., complement fixation), or may be localized to the target cells by the affinity reagent (e.g., immunotoxin, such as ricin B chain).

It is preferred that bone marrow-derived cells are collected and processed using sterile instruments and techniques, to avoid infectious complications in the recipient. Such techniques are well known in the art. The bone marrow-derived cells administered to the subject may be, or may not be, genetically engineered to produce one or more biological substances of interest, such as a neuronal factor or neurotransmitter. Genetically modified bone marrow-derived cells are utilized when augmentation of the properties of neurons derived from the bone marrow-derived cells is desired or when the production of secreted factor(s) (e.g., a neurotrophic or gliotrophic factor) in the CNS or PNS is desirable, although in certain embodiments, such as Parkinson's disease (and its subtypes) and Parkinsonism, the use of bone marrow-derived cells which have not been genetically modified to produce L-DOPA or dopamine is contemplated. Generally, a construct encoding a molecule (often an enzyme or structural protein) that is desirable in the disorder to be treated is introduced into the bone marrow-derived cells. The construct may employ a ubiquitous promoter (beta-actin, for example), but neuron-specific promoters, such as the promoters for NeuN (neuronal nuclei), Calmodulin-dependent Protein Kinase II (CaMKII), Calmodulin-dependent Protein Kinase IV (CaMKIV), any of the neurofilaments (including the 200 kD, 160 kD, 150 kD, 145 kD, 70 kD, and 65 kD forms), class III beta-tubulin calbindin D-28k, microtubule associated protein 2, synaptic protein SNAP-25, synaptophysin, NMDA receptor, neuron specific enolase, tyrosine hydroxylase, neural nestin, synapsin-1, tau, Hu, doublecortin, and the like, are preferred. For example, when the bone marrow-derived cells are utilized for the treatment of Parkinson's disease, the cells may be modified to express the enzymes necessary for dopamine production (e.g., tyrosine hydroxylase; Wolff et al., 1989, Proc. Natl. Acad. Sci. USA 86(22):9011-9014, and/or L-DOPA decarboxylase, Scherer et al., 1992, Genomics 13(2), 469-471). See, for example Gage et al. (1987, Neuroscience 23:795-807). Other examples include differentiation of marrow-derived cells into GABA-containing neurons in the basal ganglia to replace those lost in patients with Huntington's disease, and production of cholecystokinine by marrow-derived cells implanted into the temporal cortex or hippocampus to treat schizophrenia.

Introduction of genetic constructs into bone marrow-derived cells can be accomplished using any technology known in the art, including calcium phosphate-mediated transfection, electroporation, lipid-mediated transfection, naked DNA incorporation, electrotransfer, and viral (both DNA virus and retrovirus mediated) transfection. Methods for accomplishing introduction of genes into cells are well known in the art (see, for example, Ausubel, id.).

As will be apparent to one of skill in the art, it may be desirable to subject the recipient to an ablative regimen prior to administration of the bone marrow-derived cells. Ablative regimens typically involve the use of gamma radiation and/or cytotoxic chemotherapy to reduce or eliminate endogenous hematopoietic cells, such as circulating white cells and/or hematopoietic stem cells and precursors. A wide variety of ablative regimens using chemotherapeutic agents are known in the art, including the use of cyclophosphamide as a single agent (50 mg/kg q day.times.4), cyclophosphamide plus busulfan, the DACE protocol (4 mg decadron, 750 mg/m.sup.2 Ara-C, 50 mg/m.sup.2 carboplatin, 50 mg/m.sup.2 etoposide, q 12 h .times.4 IV), and the like. Additionally, gamma radiation may be used (0.8 to 1.5 kGy, midline doses) alone or in combination with chemotherapeutic agents. In accordance with standard practice in the art, when chemotherapeutic agents are administered, it is preferred that the be administered via an intravenous catheter or central venous catheter to avoid adverse affects at the injection site(s).

In certain embodiments, the bone marrow-derived cells are administered to a subject having a neuronal deficiency. Those of skill in the art (i.e., medicine, surgery, and psychiatry) will recognize subjects having neuron deficiencies, as described herein, using techniques known in the art.

Neuronal deficiency may include loss of a memory function such as, amnesia. Amnesia is an inability to recall information that is stored in the memory. There are three types of memory affected by amnesia including immediate memory, intermediate memory and long term memory. When the immediate memory is affected the patient has difficulty recalling the events that occurred in the preceding few seconds. Intermediate memory is affected when the patient cannot recall events that happened from within a few seconds to a few days prior to the cause of the amnesia. With long term memory loss the patient will be unable to recall events that occurred further back in time. Examples of memory functions include “episodic” memory (memory for events) and “semantic” memory (memory for facts) which can be lost when the memory system is damaged. Memory functions may also be classified as sensory memory, short-term memory and long-term memory.

Bone marrow-derived cells are preferably formulated in a physiologically acceptable solution (e.g., normal saline, buffered saline, or a balanced salt solution) and administered to the subject by vascular administration (e.g., intravenous infusion), in accordance with art accepted methods utilized for bone marrow transplantation. Typically, an infusion catheter is inserted into a vein, and a single cell suspension of bone marrow-derived cells is infused into the recipient subject. Preferably, the bone marrow-derived cells are administered into a peripheral vein, more preferably a superficial peripheral vein, but central venous administration (e.g., through a central venous catheter) is also contemplated. Additionally, cells may be administered by direct injection into the CNS (brain or spinal cord) or by intrathecal injection or infusion, although these routes are less preferred. It is preferred that the catheter or needle used for administration be relatively large gauge (e.g., larger than about 20 gauge) to avoid blockage of the catheter or needle by any clumps of sells present in the bone marrow-derived cell preparation.

An effective amount of bone marrow-derived cells are administered to the recipient. Preferably, at least about 10² and less than about 10⁹ cells are administered to the recipient. The number of bone marrow-derived cells administered may range from about 10¹, 10², 5×10², 10³, 5×10³, 10⁴, 10⁵, 10⁶, 10⁷, or 10⁸ to about 5×10², 10³, 5×10³, 10⁴, 10⁵, 10⁶, 10⁷, 10⁸, or 10⁹, where the upper and lower limits are selected independently, except that the lower limit is always less than the upper limit. The number of cells administered will depend on both the neuronal deficiency to be treated as well as the level of fractionation of the bone marrow-derived cells. As will be apparent to one of skill in the art, the number of cells necessary to form an “effective amount” decrease as the degree of fractionation or purity increases.

The bone marrow-derived cells may be delivered in a single administration or in multiple (i.e., greater than one) administrations. When the bone marrow-derived cells are delivered in multiple administrations, the spacing of the multiple administrations may be uniform or varying, but the various administrations are preferably at least one day apart, and may be separated by at least 2, 3, 4, 5, 7, 9, 11, 14, 21, 28, or more days.

In some instances, bone marrow-derived cells are administered in conjunction with one or more neuronal factors affecting the proliferation, differentiation and/or survival of neurons. Neuronal factors are well known in the art, and include nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene (implicated in X-linked Kallman's syndrome), insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF-1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-.beta. 1, -.beta.2, -.beta.3, -.beta.4, and -.beta.5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies). As will apparent to those of skill in the art, biologically active fragments and peptide mimetics may be used in addition to or instead of full length neuronal factors. Additionally, the bone marrow derived stem cells may be genetically engineered (e.g., by any DNA transformation, viral transduction, or any other genetic transduction technique known in the art) to produce the neuronal factor(s) themselves.

In certain embodiments, the neuronal factor(s) are produced by additional cells. Cells which endogenously produce a neuronal factor or which have been genetically modified to produce a neuronal factor may be used. The additional cells may be mixed with the bone marrow-derived stem cells prior to or at the time of administration, or they may be administered separately.

Additionally or alternatively, bone marrow-derived cells may be engineered to express receptors for neuronal factor(s) such as trkA, trkA[EI] (extracellular 6-amino-acid insert), trkb, trkB[T1], trkB[T2], trkC, trkC[TK+14] (14-amino-acid kinase insert), trkC[TK+25] (25-amino-acid kinase insert), trkC[TK+39] (39-amino-acid kinase insert), trkC[TK−158] (158-amino-acid deletion), trkC[TK−143] (143-amino-acid deletion), trkC[TK−113] (113-amino-acid deletion), trkC[TK−108] (108-amino-acid deletion), or p75-LNTR (low-affinity neurotrophin receptor or low-affinity NGF receptor).

The neuronal factor may be admixed with the bone marrow-derived cells or administered separately. When the neuronal factor is administered separately from the bone marrow-derived cells, the neuronal factor may be administered systemically (e.g., by parenteral administration, such as IV, subcutaneous, intramuscular, or intraperitoneal), but is preferably administered to the nervous system (e.g., by direct injection into the brain or spinal cord or by intrathecal injection/infusion). Administration of the neuronal factor may be by bolus or by infusion.

Alternately, or in addition to the administration of a neuronal factor, tissue damage may also be used to induce the endogenous production of neuronal factors at either the target site or a different site. Tissue damage may be produced by any means convenient, most commonly by directly creating mild physical damage at sites in the nervous system using a probe, needle, catheter, or the like.

In certain embodiments, administration of bone marrow-derived cells to a subject results in the formation of bone marrow derived neurons, derived from the bone marrow-derived cells, in the nervous system of the patient. Administration of bone marrow-derived cells results in an improvement, stabilization, or a reduction in the rate of progression of symptoms of a neuronal deficiency. The symptoms of neuronal deficiencies are well known in the art, as are methods of assessing the severity of symptoms. As will be understood by one of skill in the art, the exact symptoms will depend on the disorder and the particular patient, as neuronal deficiencies are generally pleiomorphic and follow varying natural histories in different individuals.

The invention also provides for treatment of a neuronal deficiency by administration of a bone marrow cell mobilization treatment. Bone marrow cell mobilization protocols are well known in the art. The use of granulocyte colony stimulating factor (G-CSF) for bone marrow cell mobilization is well known (see, e.g., Chao et al., 1993, Blood 81(8):2031-2035). The recombinant form of human G-CSF is commercially available as filgastrim. Recombinant human GM-CSF is also commercially available and is used in bone marrow cell mobilization protocols. Commonly used protocols involve the administration of 5-24.mu.g/kg/day of G-CSF, preferably about 10 to 12.mu.g/kg/day, for four, five or six days. GM-CSF may also be used alone, but is more preferably used in combination with G-CSF, for example in a protocol administering about 10.mu.g/kg/day of G-CSF with 5.mu.g/kg/day GM-CSF for four, five or six days (Korbling, 1999, Baillires Clin. Haem. 12(1/2):41-55).

G-CSF and/or GM-CSF may also be combined with additional agents. Flt-3 ligand (from about 1 to about 100.mu.g/kg/day) may be combined with G-CSF and/or GM-CSF. U.S. Pat. No. 5,925,568 discloses the use of MIP.alpha. for bone marrow cell mobilization. Additionally, the use of anti-VLA-4 antibody and/or an anti-VCAM-1 is disclosed in U.S. Pat. No. 5,843,438.

Additionally, a bone marrow cell mobilization therapy may be combined with the administration of bone marrow-derived stem cells for treatment of any of the disorders for which treatment by administration of bone marrow-derived stem cells is disclosed herein.

In many instances, generalized end points may be used to assess symptoms of neuron deficiencies. For example, activities of daily living (ADLs) are useful end points for assessing the integration of physical and mental function. ADLs include moving to and from bed, walking, sitting in and rising from a chair, bathing, dressing, cooking, feeding, and the like. Tests of mental function may also be of use, including the Monumental Status Examination, the Iowa Battery for the Detection of Mental Decline, the Wechsler Adult Intelligence Scale, the Wechsler Memory Scale, the Benton Visual Retention Test, the Stanford-Binet intelligence quotient examination, and the like. Additional tests useful in assessing symptoms of neuronal deficiencies include tests of directed movement, reaction time, grip and limb strength, Babinski sign, and the like. Radiological imaging may also be helpful; for example, computerized tomography (CT) and magnetic resonance imaging (MRI) scans are useful for assessing lesion number and size, and positron emission tomography (PET) scans may be used to assess functionality of particular portions of the nervous system. Combinations of selected assays are useful for particular disorders. For example, tests such as ADLs, directed movement and reaction time are useful in assessment of PD, ADLs, grip and limb strength, and Babinski's sign are useful in assessment of ALS, and ADLs, grip and limb strength, and CT or MRI scanning are useful in assessment of MS. Electrophysiological methods such as tests of nerve conduction, electroencephalograms, and the like may be used to assay nerve function. Additionally, clinimetric scales are also useful for assessment of the symptoms of neuronal deficiency disorders. Clinimetric scales are useful for quantification of overall health (e.g., Karnofsky performance score) and for symptoms of specific disorders. For example, the Chalfont Seizure Severity Scale and the Liverpool Seizure Severity Scale (Duncan et al., 1991, J. Neurol. Neurosurg. Psychiatry 54:873-876; Baker et al., 1991, Epilepsy Res. 8:245-251) are useful for measurement of epilepsy symptoms, while the Pourcher and Barbeau ataxia rating scale (1980, Can. J Neurol. Sci. 7:339-344) is useful in assessing symptoms of ataxia.

### EXAMPLES

### Example 1

### Identification of Bone Marrow-Derived Cells in the CNS

Bone marrow-derived cells were sterilely harvested from C57B/6 mice which had been modified to produce green fluorescent protein (GFP) in every cell. Marrow was harvested by flushing 2% fetal calf serum (FCS) in Hank's buffered salt solution (HBSS) through the marrow cavities of the limb bones with a 25 gauge needle. Cells were collected and suspended in 2% FCS in HBSS, filtered through 70.mu.m NITEX.RTM. (Tetko, Inc.) mesh, collected by centrifugation (approximately 400.times.g for 5 minutes), and then resuspended at 4.8×10⁷ nucleated cells/mL.

Isogeneic recipient mice were prepared for transplant by lethal irradiation (950 cGy total dose, split into equal fractions and administered 3 hours apart). The bone marrow-derived cells were administered by injection of 125 μL of the cell suspension into the tail vein.

Approximately 3 months after transplant, recipient mice were euthanized by cervical dislocation and brain cells were isolated from the recipient mice by opening the cranium, removing the brain, mincing the brains with a razor blade, rinsing the minced tissue twice with HBSS, and resuspending in 10 mL of PPD solution (2.5 U/mL papain, 250 U/mL DNase I, 1 U/mL dispase II, in HBSS plus 12.4 mM MgSO₄). The tissue was incubated at 37C for 30 minutes, then digestion was stopped by the addition of 2 mL of fetal bovine serum (FBS). The tissue was dissociated by trituration, then filtered through a 70 mm sieve (BD Biosciences) and washed 3 times with 20% FCS in Dulbecco's Modified Eagles medium (DME).

Cells were resuspended in 200 mL of 5% FBS in phosphate buffered saline (PBS) and incubated on ice for 15 minutes with Tricolor (TC)-conjugated rat anti-mouse CD 1 lb and Allophycocyanin (APC)-conjugated rat anti-mouse CD45. Control cells were incubated with isotype matched TC- and APC-conjugated specific for irrelevant antigens. The cells were washed once with 5% FBS in PBS (FBS/PBS), and resuspended in 200 mL of FBS/PBS and fixed by addition of 400 mL of solution A from the “Fix and Perm” kit (Caltag) and incubation at room temperature for 15 minutes. The cells were washed twice, then stained for nuclear DNA by incubation in FBS/PBS containing 0.12 mg/mL Hoechst 33258. Cells were analyzed using a MOFLO.RTM. flow cytometer (Cytomation, Inc.) and FLOJO™ software (Tree Star, Inc.).

A distinct population of GFP+ cells was identified in animals transplanted with GFP+ bone marrow, as compared to cells from animals transplanted with non-GFP expressing marrow. Approximately 95% of the GFP+ cells were positive for CD11b and/or CD45, markers of myelomonocytic cells and circulating white cells, respectively. Approximately 5% of the GFP+ cells were clearly negative for both hematopoietic cell markers.

In a similar experiment, GFP+ cells from dissociated brain were stained with antibodies recognizing Hu, which is a nuclear protein only expressed in neurons, and Hoechst 33258, which stains DNA. The anti-Hu antibody was detected with a secondary antibody labeled conjugated to Texas Red. The stained cells were embedded in a collagen matrix and evaluated by epifluorescence microscopy which revealed that 3% of GFP+ cells expressed the neuronal protein Hu. Further analysis confirmed that the anti-Hu staining was localized to the nuclear regions of isolated GFP-positive, bone marrow-derived cells. These finding suggested that exposure to the CNS environment may have led a subpopulation of bone marrow-derived cells to acquire a novel neuronal phenotype.

### Example 2

### Identification of Bone Marrow-Derived Neurons

Animals were prepared and transplanted with GFP+ bone marrow-derived cells as described in Example 1.8 to 12 weeks after transplant, the recipient mice were euthanized and perfused with 25 mL of 4.degree. C. phosphate buffer (pH 7.4) followed by 25 mL of 4.degree. C. 1.5% paraformaldehyde in phosphate buffer. Brains were removed and incubated in 1.5% paraformaldehyde, 0.1% glutaraldehyde, 20% sucrose in phosphate buffer overnight at 4.degree. C. The brains were embedded in TISSUE-TEK™ O.C.T. compound (Sakura Finetek) and snap frozen. 20-40 μm coronal cryosections were taken from the olfactory bulb (Bregma −4.1 to −3.6).

Sections were blocked with 25% normal goat serum (NGS), 0.25% Triton.RTM. X-100, and rat anti-mouse-CD16/32 (1:1000, Pharmingen) in PBS for one hour. The sections were stained with anti-NeuN (MAB377 from Chemicon, 1:4000), anti-200 kD neurofilament (AB1989 from Chemicon, 1:400), anti-beta3-microtubulin (TUJ1 from Covance, 1:1000) anti-glial fibrillar acid protein (GFAP; polyclonal antibody, Dako, 1:2000) or anti-F4/80 (Caltag, 1:800) antibodies for 48 hours at 4.degree. C., washed, then incubated with the appropriate secondary antibody (Goat anti-mouse and goat anti-rabbit antibodies conjugated to Texas Red or Cy5, 1:800, Molecular Probes, Inc.). The sections were imaging using a laser confocal microscope adjusted to yield optical sections with a theoretical thickness of 0.3 to 0.4 μm. Sequential laser excitation was employed to eliminate bleedthrough.

An average of 220 (SD+96) GFP+cells were observed per section of the olfactory bulb (OB). Of these GFP+ cells, the majority, 72%, expressed the F4/80 microglial surface marker. Many of the GFP+/F4/80− cells had morphologies suggestive of neuronal cells.

The morphology of GFP+ cells was analyzed by visual inspection using epifluorescence and laser scanning confocal microscopy. The majority of GFP+ cells that co-stained for neuronal markers (NeuN or NF-H) were triangular in morphology (61.7% and 60.9%, respectively), while F4/80+ cells were mostly spindle or stellate in morphology. Because neurons in the CNS often assume triangular morphology, cells with triangular morphology were subdivided into three categories: those having no observable extensions (+), those having a single observable extension of less than 10.mu.m and those having either a single observable branched extension or more than one observable extension (+++). Results of the morphological analysis are summarized in Table 1.

TABLE 1

| | | Markers | | |
|---|---|---|---|---|
| | | NeuN+ | NF-H+ | F4/80 |
| Morphology | | (n = 165) | (n = 129) | (n = 229) |
| Triangular | + | 13.3% | 34.6% | 18.0% |
| | ++ | 34.4% | 18.9% | 3.5% |
| | +++ | 14% | 7.4% | 3.5% |
| Round | | 12.3% | 9.4% | 0 |
| Oval | | 8.6% | 10.9% | 3.6% |
| Rod | | 2.5% | 0 | 0 |
| Spindle | | 1.2% | 10.9% | 35.7% |
| Stellate | | 2.5% | 2.3% | 32.1% |
| Other | | 11.1% | 2.4% | 3.6% |

Sections of the OB were also analyzed with respect to localization of bone marrow-derived cells. Coronal sections of the OB were stained for a single marker and analyzed with respect to GFP+ cells in each layer of the OB. 12 sections, averaging 10,400 (.+−0.600) neurons per section, were analyzed for localization of GFP+ neurons (8 for NeuN+ cells, 4 for NF-H+ cells), 4 sections, averaging 2000 (.+−0.200) astrocytes per section, were analyzed for localization of GFP+ astrocytic cells, and 3 sections, averaging 550 (.+−0.50) microglia per section, were analyzed for localization of GFP+microglial cells. The majority of GFP+ cells were found in the superficial axon layer (SAL), and relatively large numbers of GFP+ cells were also found in the glomerular layer. Interestingly, no GFP+ cells expressing an astrocytic marker (glial acid fibrillar protein, GFAP) were identified in any of the sections, contrary to previously published reports (Eglitis et al., id.). The results of the anatomic analysis of the OB are summarized in Table 2.

TABLE 2

GFP+ Cells

| | | |
|---|---|---|
| Neurons | Astrocytes | Microglia |

| Layer | NeuN+ | NF-H+ | GFAP+ | F4/80+ |
|---|---|---|---|---|
| Superficial | 105 | 66 | 0 | 312 |
| Axon | | | | |
| Glomerular | 30 | 41 | 0 | 114 |
| External | 14 | 10 | 0 | 56 |
| Plexiform | 4 | 0 | 0 | 7 |
| Mitral Cell | | | | |
| Internal | 0 | 0 | 0 | 8 |
| Plexiform | 12 | 11 | 0 | 13 |
| Granule | | | | |
| TOTAL | 165 | 129 | 0 | 510 |

### Example 3

### Bone Marrow Derived Purkinje Cells

These experiments demonstrate that bone marrow-derived cells cross the blood-brain barrier and contribute to neurons, particularly Purkinje cells, in the CNS of human patients. Purkinje neurons are generated only during early brain development. In humans, generation of Purkinje neurons starts at 16 weeks of gestation and is complete by the end of the 23rd week. Most of the maturation of the characteristic dendritic trees of human Purkinje neurons is finalized during the first year of life. By contrast to other neurons in the adult brain, there is no evidence for the generation of new Purkinje neurons after birth, even in cases of severe Purkinje cell loss caused by trauma or genetic disease.

The human brain contains 15 million Purkinje cells, which are among the largest neurons in the CNS. A typical Purkinje neuron has >50-fold the volume of neighboring neurons in the brain, and its complex dendritic extensions receive inputs from as many as one million granule cells. Purkinje cells play vital roles in maintaining balance and regulating movement. A loss of Purkinje cells results in deficits in these functions in several disorders: ataxia-telangiectasia, the most common cause of progressive ataxia in infancy; Menkes' Kinky Hair syndrome; the alcoholic cerebellar degenerations, particularly Wernicke-Korsakoff syndrome; and various prion diseases including scrapie, Creutzfeldt-Jakob, and Kuru. Thus, renewal or rescue of Purkinje neurons has significant therapeutic implications.

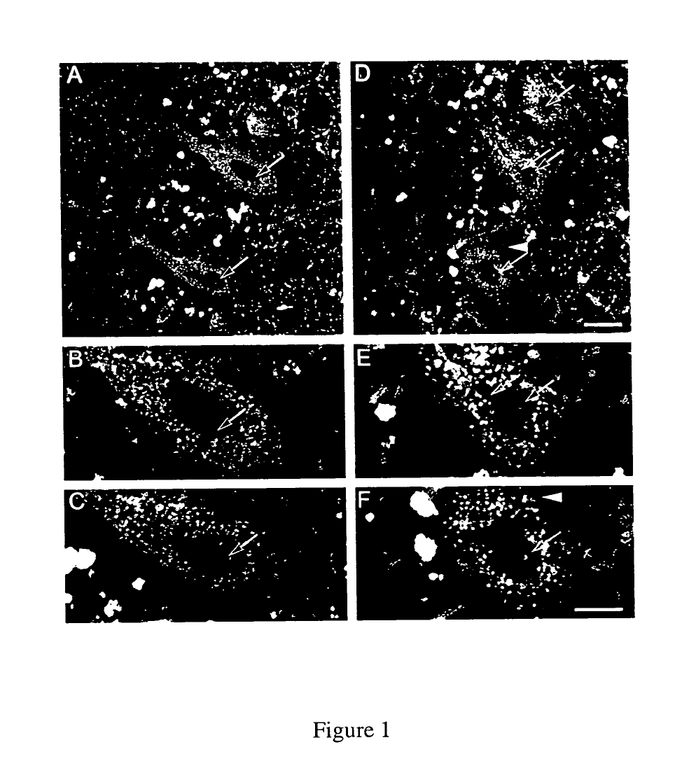

As shown in FIG. 1, in control male and female cerebellar sections processed for in situ hybridization, human X and Y chromosomes can be readily visualized with the specific, labeled probes. Large, yellow, pear-shaped Purkinje neurons are easily recognized between the cell-sparse molecular layer containing stellate and basket neurons (FIG. 1 Left) and the inner granular layer (FIG. 1 Right), composed primarily of small granule neurons and a few Golgi neurons (FIGS. 1A and D). The characteristically large size of the Purkinje cells and thick dendritic projections that extend into the molecular layer are readily apparent. Nuclei of Purkinje cells, visualized as blue when stained with To-Pro-3, have typical diffuse chromatin and a distinctive large nucleolus, whereas the nuclei of the neurons in the surrounding granular layer have very little cytoplasm, small nuclei with densely packed chromatin and no obvious nucleolus. Thus, these cell types are easily distinguished by histology after in situ hybridization without the need of antibody staining, an assay precluded by the digestion procedure.

In situ hybridization revealed that X and Y probes yielded red and green signals that clearly distinguished the two sex chromosomes by confocal microscopy. The Vysis X chromosome probe is conjugated to Spectrum orange that fluoresces at a peak of 588 nm (red), whereas the Y chromosome probe is conjugated to Spectrum green that fluoresces at 524 nm (green). Fortuitously, the autofluorescence in the green and red channels superimposed to yield a yellow color that allowed distinction of the Purkinje cell body cytoplasm. In cerebellar sections from control female brains, Y chromosome labeling was never detected. FIG. 1 shows labeling with both X and Y chromosome probes of sections from controls, a normal female brain (A-C) and a normal male brain (D-F). In FIG. 1A two female Purkinje cells and in FIG. 1D three male Purkinje cells are shown between the cell-sparse molecular layer (left) and the granular layer (right). Enlargements of the Purkinje cells in FIG. 1A are shown in FIGS. 1B and C, and enlargements of FIG. 1D are in FIGS. 1E and F, but without nuclear staining to enhance the visualization of the chromosomes. Note that two sex chromosomes are not always seen in every control Purkinje nucleus because of the thin sections required (FIGS. 1B, C, and F). In contrast, the nuclei of most of the smaller granule neurons exhibit staining of two chromosomes, as the entire nucleus is usually contained in the section. However, in 10-μm sections two or more granule neuron nuclei may be superimposed, giving the impression of more than two sex chromosomes per cell. It was possible to verify that each granule neuron nucleus contained only two sex chromosomes by examining individual serial optical sections within the stack. Occasionally, the X chromosome (FIG. 1B) or the Y chromosome (FIG. 1E) appears to be outside or proximal to the Purkinje nucleus, but this is caused by the projection of stacked serial confocal images. This finding was confirmed by examining individual 1-μm optical sections within the stack that are sufficiently thin to permit precise cellular localization of the chromosome (not shown). On the other hand, in some cases, a chromosome belongs to an abutting cell, which is evident from the cytoplasm separating the two cells (compare FIGS. 1D and F, white arrowhead). In the granular cell layer many cells can be seen with one X and one Y chromosome. Because these cells are small and densely packed with little cytoplasm, it is often difficult to distinguish the borders between adjacent cells, a problem not encountered with Purkinje neurons because of their large size and abundant cytoplasm.

Cerebellar tissue samples obtained at autopsy were analyzed from female patients with hematologic malignancies. Initially, chemotherapy was accompanied in most patients by total body irradiation to reduce the malignant cell population and decrease rejection of donor cells. In a few cases marrow cells from male donors were then infused into female patients, whereas most received sex-matched bone marrow. Immunosuppressive agents were given to decrease graft-versus-host reactivity. The four subjects of the study were selected based on the following criteria: sex (male donor and female recipient), availability of brain tissue, survival for 3-15 months posttransplant, and death unrelated to CNS complications. Five female patients transplanted from female donors were chosen as controls by using the same criteria. Cerebellar tissue sections were cut and coded to ensure patient anonymity and “blinded” analysis.

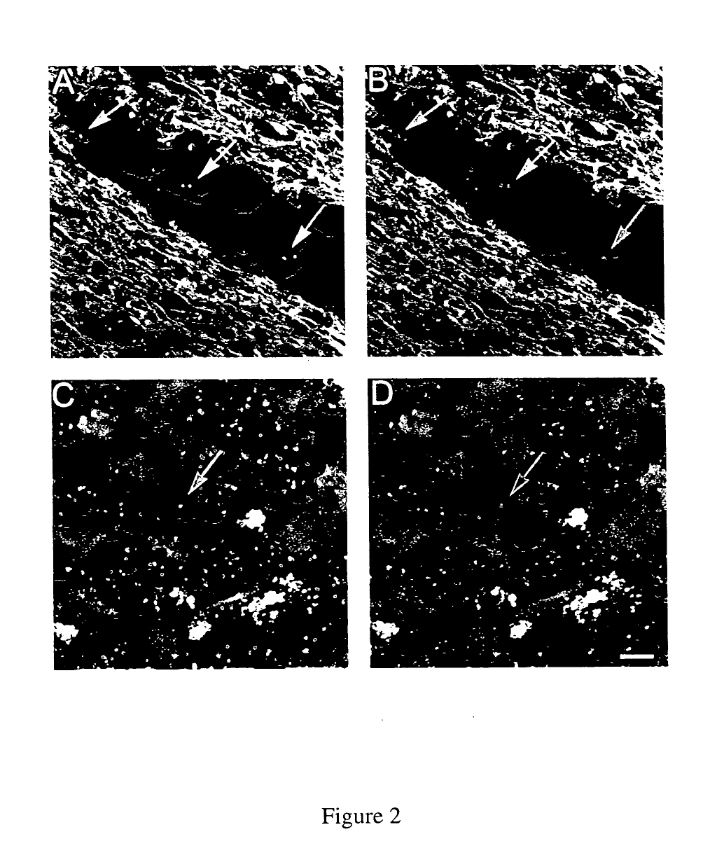

Examination of cells within blood vessels and parenchyma of cerebella underscored the high degree of specificity of the Y chromosome probe. In sections from all of the sex-mismatched transplant patients, Y and X chromosomes were found in numerous cells, presumably blood cells, within the lumina of cerebellar vessels (FIGS. 2A and B). Variation among patients may have resulted from differing degrees of hematopoietic reconstitution that was not determined years ago when these patients died and could no longer be ascertained. In sex-mismatched transplant patients, an occasional male donor-derived cell (Y chromosome in nucleus) was found in the granular cell layer (FIGS. 2C and D), whereas a Y chromosome was never found in the granular cell layer of female patients who received a bone marrow transplant from a female donor. Cells in the parenchyma are likely to be macrophages and microglia that are well known to be derived from bone marrow (12-14). Because of the inability to perform immunohistochemistry on these highly digested tissues, the specific identity of these cells could not be discerned, because unlike Purkinje cells, their morphology was not distinct.

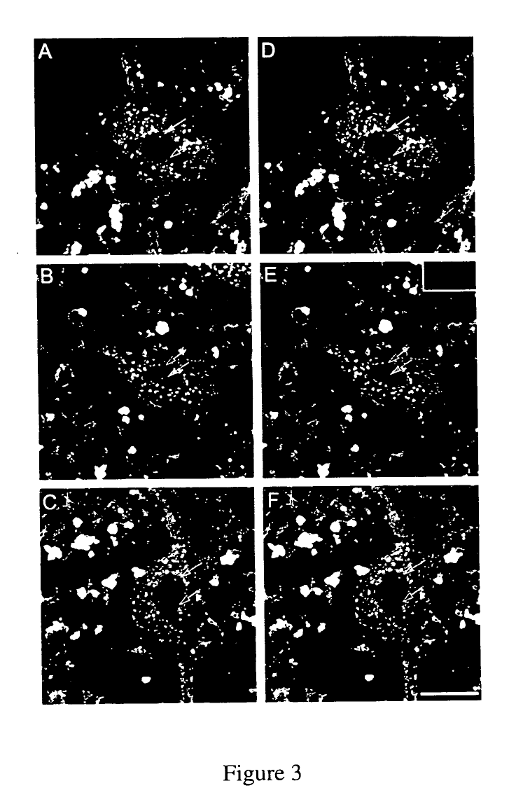

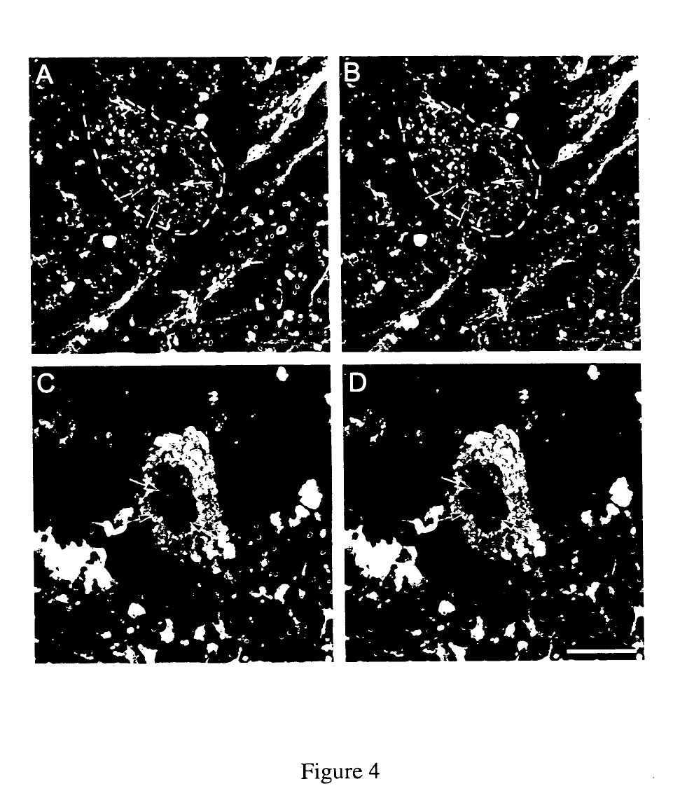

Male chromosomes were readily detected by epifluorescence in the relatively thin sections of Purkinje neurons from female brains. Following along the border of the dendritic layer, each Purkinje cell was examined for the presence of a green-labeled Y chromosome, and those with Y chromosomes were then imaged at high resolution with the confocal scanning laser microscope. Y chromosomes were found in four of the total 5,860 Purkinje nuclei examined by epifluorescence in sex-mismatched transplant patients (FIGS. 3 and 4A and B). No Y chromosomes were found in Purkinje nuclei from sex-matched transplant patients (controls). In rare cases, the X chromosome assumed a dumbbell configuration, as seen in FIG. 3E (see Inset). The distance between the two red spots in 14 different X chromosomes that had dumbbell shapes averaged 1.1±0.3 μm and the greatest distance between two such spots was 1.9 μm. Dumbbells were not caused by radiation and bone marrow transplantation as they are routinely observed in normal cells, as discussed in the Vysis protocol booklet, in which criteria are provided to distinguish a single chromosome with a dumbbell shape from two distinct chromosomes. Analysis of distances between the two red spots allowed distinction of whether such signals derived from one (FIGS. 3B and E) or two (FIG. 4) chromosomes (see below).

In two of the Purkinje cells analyzed, three sex chromosomes were observed within the same Purkinje nucleus (FIG. 4). In one case, a Y chromosome was detected together with two X chromosomes in a serial stack of optical confocal images (FIG. 4A). In another case, one of the randomly scanned Purkinje cells was found to contain three X chromosomes (FIG. 4B). No dumbbells were evident. Indeed, the closest chromosomes in the cells with three chromosomes were >4.0 μm apart. Thus, it is highly unlikely that the probe bound parts of a single chromosome. Notably, the finding of these two cells with more than a diploid sex chromosome composition raised the possibility that the contribution of donor-derived bone marrow cells to the Purkinje neuron population might occur by fusion of these two cell types.

Although the possibility remains that the Purkinje cells with one X and one Y chromosome arose de novo from cells within the bone marrow, an argument based on sampling can be made in support of cell fusion. Each of the cells in FIG. 3 contain only two sex chromosomes, which might suggest that they resulted directly from a male stem cell present in the bone marrow that changed to become a Purkinje neuron in the brain. On the other hand, because less than half of a Purkinje cell nucleus was encompassed in the sections analyzed, it is quite possible that our analyses did not include all sex chromosomes present in a given Purkinje cell. Nonetheless, whenever a Y chromosome was detected, an X chromosome was also present. To address the possibility that the sex chromosomes were underrepresented in our sample, the probability of observing zero, one, or two chromosomes in a section containing less than half of a Purkinje cell nucleus was determined. A total of 214 randomly scanned cells were selected and analyzed for the number of sex chromosomes they contained by reconstructing a series of optical sections obtained from confocal images. The results revealed the following frequencies of X chromosomes in optical sections: 32% contained zero, 46% contained one, and 21% contained two chromosomes. Thus, in diploid cells only one-fifth of randomly sampled 10-μm sections of Purkinje nuclei exhibited the full complement of sex chromosomes. As a result, the finding of an X and a Y chromosome in the same partial Purkinje cell nucleus may well underestimate the total number of sex chromosomes in that cell. In addition, the low frequency of a diploid chromosome content suggests that detection of three chromosomes would occur in less than one-fifth of all cells analyzed and that the probability of detecting four chromosomes would be exceedingly low. Taken together, this analysis and the data indicate that cell fusion occurred.

The data presented show that adult human bone marrow cells can contribute to mature Purkinje neurons in adult women with hematologic malignancies. Even though this is not a frequent event (0.1% of the cells examined), it is surprising that it occurs at all because the generation or repair of these cells after birth had not been documented (2, 6-9). Because there are 15 million Purkinje cells in the human adult brain, by extrapolation, the total number of cells affected by a bone marrow transplant could be quite substantial.

Methods

Tissue Specimens.

At death, brains were removed and fixed intact in neutral buffered formalin (3.7-4.0% formaldehyde) for 10-14 days. Tissue blocks were then embedded in paraffin. For this study, 10-μm sections were cut from cerebellar tissue of each transplant patient and from untransplanted male and female control brains and mounted onto glass slides. Only half of a Purkinje cell nucleus could be included in these 10-μm sections; thicker sections could not be used because Y chromosomes could not be identified by epifluorescence before in-depth confocal analysis.

In Situ Hybridization.

Paraffin was removed from sections with three changes of xylene (10 min each), rehydrated through graded alcohols (3 min each), and washed twice with double distilled water (ddH2O). Sections were placed in 0.2 M HCl at room temperature for 15 min and rinsed twice in ddH2O and once in Tris-EDTA. Sections were then digested in Proteinase K (3.8 μg/ml Tris-EDTA) at 37° C. for 37 min and rinsed twice with ddH₂O and once with 2×SSC. Slides were placed in preheated pretreatment solution (sodium isothiocyanate, Vysis, Downers Grove, Ill.) at 82° C. for 37 min followed by three rinses at room temperature with 2×SSC. Sections were digested in protease I (pepsin) 4 mg/ml in protease I buffer (Vysis) for between 10 and 37 min (the time differing depending on fixation of sample), followed by three rinses in 2×SSC. Sections were denatured for 5 min at 73° C. in 49 ml of formamide (fresh or frozen aliquots)/7 ml of 20×SSC/14 ml of ddH₂O, then dehydrated through a graded series of ethanols. A CEP XY DNA probe (Vysis) was applied to each section, sealed under a glass coverslip, and incubated overnight at 42° C. The Vysis probes detect the alpha satellite sequences in the centromere region of the X chromosome (DXZ1 locus) and the satellite III heterochromatin DNA at the Yq12 region of the Y chromosome (DYZ1 locus) (see Vysis). The next day, coverslips were removed in 2×SSC, rinsed for 2 min in 2×SSC/0.1% Nonidet P-40 at 73° C., allowed to air dry, and mounted in DAPI II mountant (Vysis) to which To-Pro-3 iodide (Molecular Probes) was added at a dilution of 1:3,000.

Microscopy.

Cerebellar sections were viewed at ×63 by using a Zeiss LSM510 laser scanning confocal microscope equipped with epifluorescence. The margin between the granular cell layer and the acellular molecular layer was scanned for Purkinje cell bodies and the presence of a Y chromosome by using epifluorescence. The green Y chromosomes were evident as a lime-green dot in the midst of the yellow-green autofluorescent cytoplasm. A total of 5,860 Purkinje cells from sex-mismatched bone marrow transplants and 3,202 Purkinje cells from sex-matched transplants were counted and assessed for the presence of a Y chromosome (green, visible by epifluorescence), allowing a determination of the overall frequency of Y chromosome-containing cells. As part of the blind study, images of every 20th Purkinje neuron were scanned on the confocal microscope by acquiring 1-μm serial optical sections through the portion of the nucleus present in the section (<50%). The stacks of images were then used for further analysis of the sex chromosome content of Purkinje cells in general and the frequency of zero, one, and two sex-chromosomes within nuclear sections of the size analyzed here. From all of the control and test Purkinje cells serially scanned and reconstructed, a total of 214 nuclei were used to assess the average number of sex chromosomes in randomly sampled Purkinje neurons.

### Example 4

### Bone Marrow Derived Cells Fuse with Purkinje Cells

As described herein, BMDCs can contribute to the regeneration of neural tissue. Experiments described in this example demonstrate that, in the case of Purkinje cells, the contribution of BMDCs to neural tissue occurs by fusion of BMDCs with neurons to produce stable heterokaryons. The previously unrecognized finding that binucleate, chromosomally balanced heterokaryons are produced in vivo in tissues such as brain is remarkable, as stable heterokaryons were only thought to occur artificially in tissue culture. In these in vivo heterokaryons, the neurons were dominant over the BMDCs, as no mitosis was evident and the morphology was typical of functional Purkinje neurons, with complex dendritic trees and axons. Moreover, cytoplasmic factors within the Purkinje neurons reprogrammed the fused BMDC nuclei resulting in nuclear swelling, decondensed chromatin and activation of a Purkinje neuron-specific transgene, L7-GFP.

Purkinje neurons are mononucleate diploid cells that are generated only during gestation and not replaced after loss through trauma or genetic disease. The complexity and importance of the Purkinje neuron is underscored by the fact that the axons of the Purkinje neurons are the only efferent from the cerebellum to other brain regions, and in humans each Purkinje neuron can receive over 1 million inputs from other neurons. Indeed, these large, highly specialized, Purkinje neurons of the cerebellum are critical to balance and fine motor control, and defects in these cells result in ataxias.

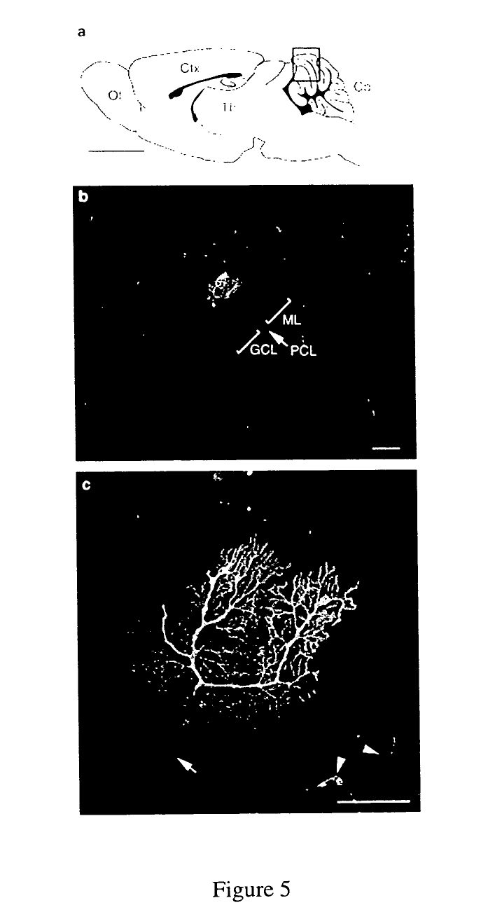

To elucidate the mechanism by which BMDCs contribute to neural tissue, the bone marrow from transgenic mice ubiquitously expressing GFP was harvested and transplanted by tail-vein injection into lethally irradiated syngeneic recipient mice. Several months later, Purkinje neurons expressing GFP were detected in the cerebella of recipient animals (FIG. 5). These GFP-positive Purkinje cells were indistinguishable from normal Purkinje neurons, with their soma in the Purkinje cell layer (PCL) and a large, apical and highly branched dendritic tree that extended into the cell-sparse molecular layer (ML; FIG. 5b). The single axon from the Purkinje neuron extended through the granular cell layer (GCL) into the white matter and was the only output axon from this neuron to other brain regions. An image of a bone-marrow-derived Purkinje neuron at low magnification shows this cell in the context of a cerebellar lobe (FIG. 5b). At higher magnification, laser-scanning confocal microscopy reveals part of the descending axon (FIG. 5c, arrow) and many small synaptic spines on the extensive dendritic tree. Other GFP-positive BMDCs, such as microglia and macrophages, were readily apparent throughout the brain (FIG. 5b); two such cells are marked with arrowheads in FIG. 5c. The architecture and structure of the dendritic tree of the GFP-positive Purkinje cell with its many synaptic spines are indistinguishable from typical Purkinje neurons and are characteristic of healthy functioning neurons.

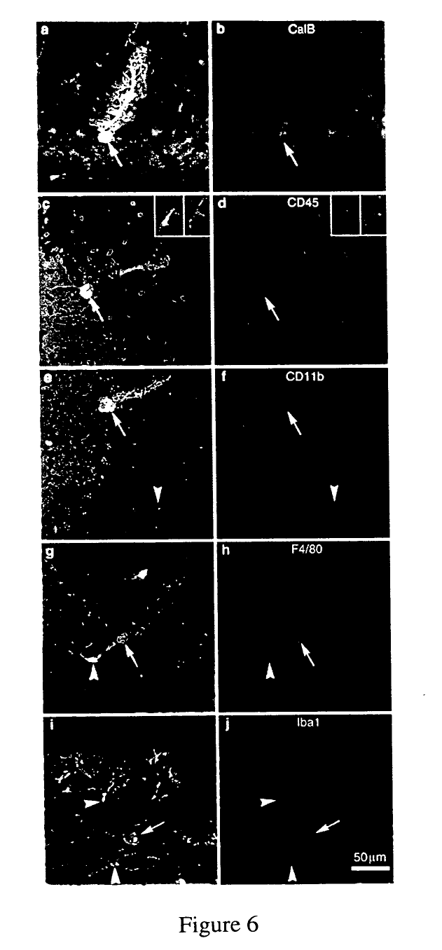

Applicants investigated whether GFP-positive Purkinje neurons expressed genes that are typically found in Purkinje cells, bone marrow cells, or both. When analysed by immunofluorescence microscopy, all of the GFP-positive Purkinje neurons strongly expressed the calcium-binding protein, calbindin, a hallmark of the Purkinje cell type (FIGS. 6a, b). No other cell type expressed calbindin in the cerebellum. To assess whether they still expressed markers typical of bone marrow cells, GFP-positive Purkinje neurons were assayed for haematopoietic markers. Sections containing BMDC Purkinje neurons were stained with antibodies against CD45 (a pan-haematopoietic marker), CD11b (a macrophage/microglia marker), F4/80 and Iba1 (microglial markers; FIGS. 6c-j). The GFP-positive Purkinje neurons were negative for all four of these haematopoietic markers, suggesting that the genes encoding these products were either inactivated or never expressed in the BMDCs that resulted in the GFP-positive Purkinje neurons in the brain. The BMDCs also yield other cell types in the cerebellar parenchyma, including GFP-positive microglia and macrophage cells. As expected, these GFP-positive BMDCs expressed haematopoietic markers (insets in FIGS. 6c, d; arrowheads in FIGS. 6e-j). Thus, co-expression of Purkinje neuron gene markers and haematopoietic markers was not observed.

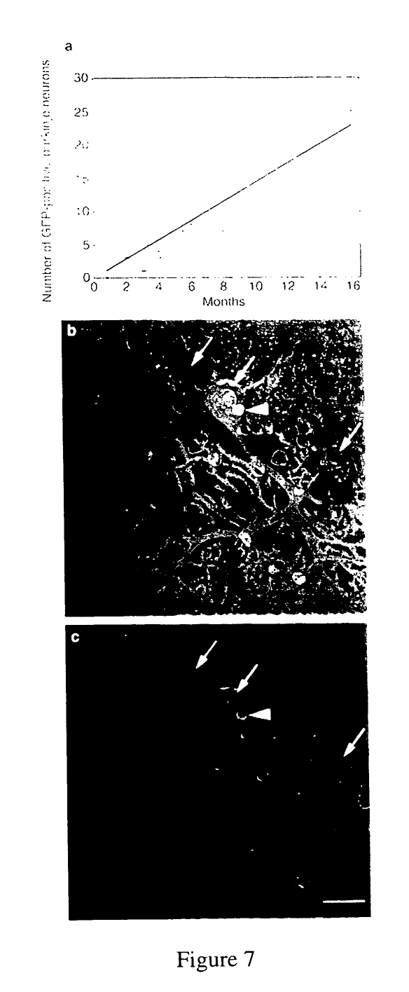

Applicants then determined the time course of BMDC contribution to the Purkinje cell pool. Mice were transplanted with bone marrow at two months of age and the number of GFP-positive Purkinje neurons detected in 20 mice under non-selective conditions was scored over a period spanning 1.5 years, approximately 75% of the average mouse lifespan (FIG. 7a). GFP-positive neurons were not apparent until several months after transplantation, and the maximum number observed under these non-selective conditions was 60 neurons in one animal after 1.5 years. A linear increase in GFP-positive neurons was observed that correlated with age, a pattern that was statistically significant up to 16 months after transplantation.

Applicants analysed the nuclear composition of the GFP-positive Purkinje neurons to determine whether they arose de novo from BMDCs or through fusion to endogenous Purkinje neurons. Using a laser-scanning confocal microscope, serial 1 μm optical sections were obtained through the entire cell body of GFP-positive Purkinje cells. Serial reconstruction of these cells revealed that in the more than 300 cases where it was possible to image the full extent of the soma, two nuclei were always detected. A typical GFP-positive Purkinje neuron with an axon exiting the soma from the top right and a primary dendrite with several secondary and tertiary dendrites is shown (FIGS. 7b, c). As with all of the GFP-positive Purkinje neurons, numerous small synaptic spines (the post-synaptic specializations of active synapses) were readily apparent on the dendrites. The endogenous Purkinje nucleus of the recipient was enlarged, with dispersed chromatin and a prominent nucleolus (FIGS. 7b, c; middle arrow), similar to other neighboring Purkinje nuclei evident in this field of view (FIGS. 7b, c; left and right arrows). By contrast, the putative bone-marrow-derived nucleus that fused into the host Purkinje neuron contained compact highly condensed chromatin (FIGS. 7b, c arrowhead). These results indicate that BMDCs contribute to the Purkinje neurons by fusion and not by de novo neurogenesis.

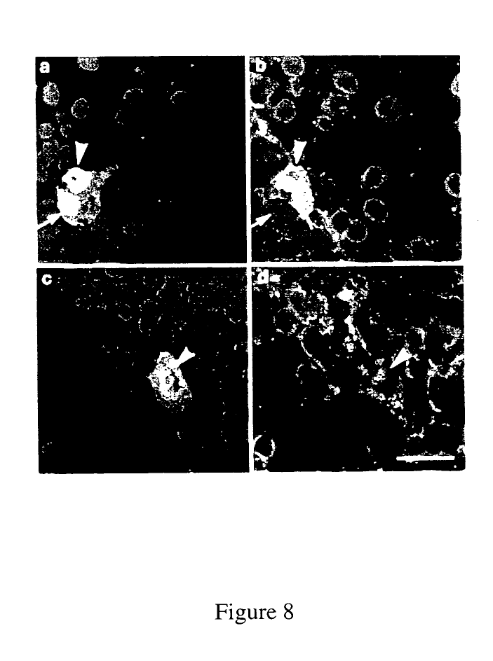

To determine definitively the cellular origin of each nucleus within GFP-positive Purkinje neurons, bone marrow from male donor mice was transplanted into female recipient mice using the same experimental paradigm described above. The presence of a male nucleus was assayed using a Texas-Red-labeled DNA probe specific to the Y chromosome and examination by fluorescence in situ hybridization (FISH). One year after transplantation, brains were sectioned at 12 μm and serial cerebellar sections were stained for GFP and counterstained with the nuclear stain To-Pro3 to visualize nuclear DNA. Serial 1 μm optical sections were obtained to determine the number of nuclei in GFP-positive cells. A motorized stage was used to record the x, y and z coordinates, ensuring the precise relocation of GFP-positive cells after the proteinase K digestion and FISH staining protocol, which removes most of the GFP staining. Representative examples of GFP-positive Purkinje neurons with two distinct To-Pro3-labelled nuclei are shown (FIGS. 8a, c). After FISH, a red Y-chromosome was detected in one of the two nuclei in each cell (FIGS. 8b, d; arrowhead). The two sets of panels in this figure show cells in the same locations before and after the extensive proteinase K digestion of the tissue that is necessary for FISH. The other nucleus within the GFP-positive soma (FIG. 8b, arrow) is the endogenous cell nucleus of the Purkinje neuron that does not contain a Y-chromosome. In the example shown in FIGS. 8c, d, the GFP-positive soma was found in two adjacent sections. The chromatin in the donor-derived Y-chromosome-positive nucleus of this cell was as dispersed as the host nucleus, with a prominent nucleolus; a structure not seen in the compact chromatin characteristic of marrow-derived cells. Donor-derived microglial cells were evident in the host tissue, and these cells also contained a Y chromosome. Despite the change in nuclear morphology, there was no evidence of cytokinesis or karyokinesis in any of the cells analysed. Indeed, the fused cells seemed to be stable heterokaryons that persisted over time. Furthermore, there was no evidence of GFP-positive Purkinje neuron death, such as blebbing or membrane fragmentation, among the more than 300 GFP-positive neurons examined. These data indicate that the GFP-positive Purkinje neurons found in the host cerebellum are the result of fusion between a host female Purkinje cell and a male BMDC.

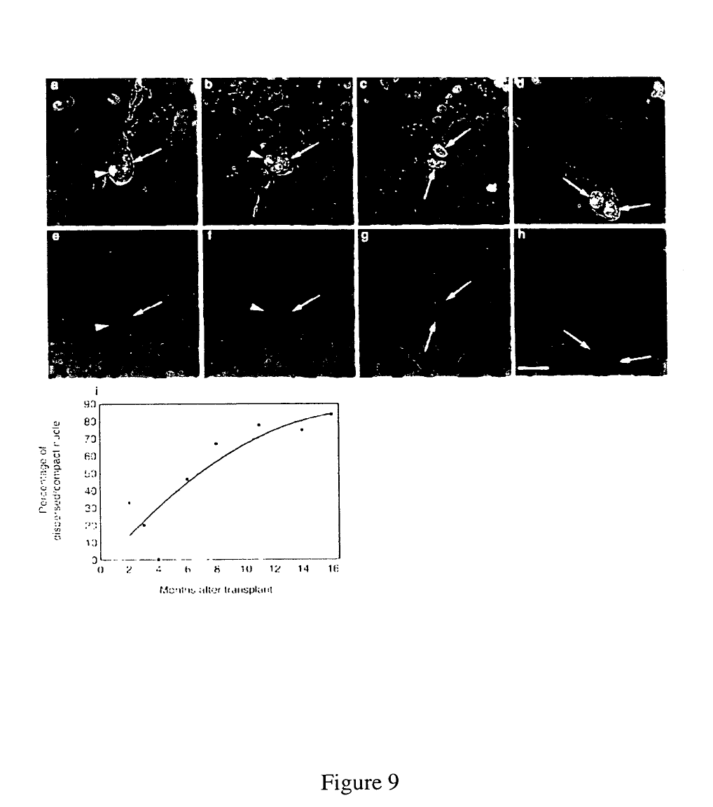

During the course of this analysis, Applicants observed that the structure of the two nuclei differed markedly among the GFP-positive Purkinje cells. Approximately 50% of the GFP-positive cells contained one large ‘Purkinje-like’ nucleus, with dispersed chromatin, and one small ‘bone-marrow-like’ nucleus, with compact chromatin. In the other cells scored, both nuclei appeared Purkinje-like. The range of nuclear morphologies that were observed is shown in FIG. 9. A time course of chromatin alteration demonstrates that the ratio of nuclei with dispersed-to-compact chromatin in the cerebella of individual mice increased over time (FIG. 9i). These data suggest that once a BMDC with a compact nucleus fuses to a Purkinje neuron (FIGS. 9a, e), the bone-marrow-derived nucleus becomes less compact and dense (FIGS. 9b, f) and finally assumes the morphology of the Purkinje nucleus to which it fused (FIGS. 8 and 9c, d, g, h). This increasing trend towards dispersed chromatin in the fused BMDC nucleus over time suggests that the fusion events are stable.

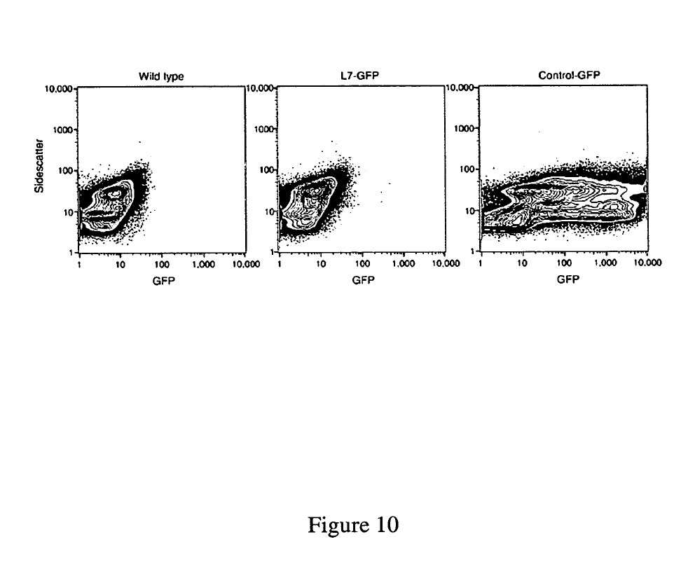

The activation of previously silent genes by intracellular signals generated by one of the two nuclei in a heterokaryon has been well established in vitro. To determine whether the changes in chromatin structure observed in the fused BMDC nuclei correlate with reprogramming and activation of Purkinje genes, a transgenic mouse that expresses GFP under the control of the Purkinje-specific promoter, L7-pcp-2 was used as a bone-marrow donor. The previously described expression pattern of the L7-GFP promoter was confirmed by analysing sections from the brain of these transgenic mice. In the brain, the only GFP-positive cells detected were the Purkinje neurons. Flow cytometry analysis of the L7-GFP bone marrow showed that these transgenic mice do not express GFP in their bone marrow. Indeed the fluorescence-activated cell sorting (FACS) plots of the L7-GFP transgenic cells were indistinguishable from those of wild-type marrow and were three orders of magnitude lower than the GFP fluorescence obtained from GFP-positive bone marrow (FIG. 10). Thus, the L7-GFP transgenic promoter is inactive in the bone marrow of this mouse line.

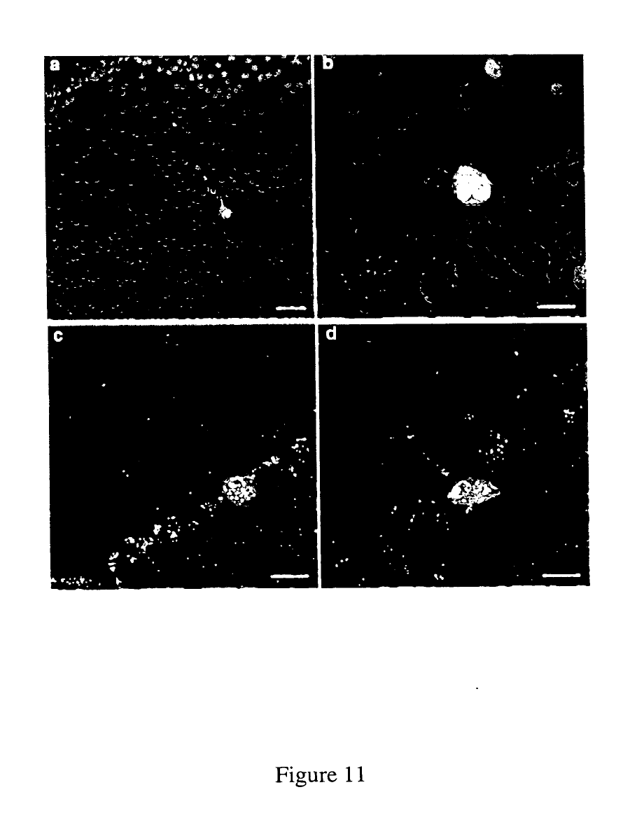

Four mice were sacrificed five months after receiving a bone marrow transplant from the L7-GFP mouse and then analysed. L7-GFP-positive Purkinje neurons were found in the cerebella of all four mice, and on average 2-3 fully mature GFP-positive neurons were observed in each mouse (FIG. 11), correlating with the prediction for five months after transplantation (FIG. 7a). All of the L7-GFP-positive Purkinje cells contained two nuclei (FIGS. 11a, b). In the cells shown in FIGS. 11c, d, one nucleus was evident in the confocal image, whereas the other was in a different plane of focus. Donor-derived haematopoietic cells such as microglia and macrophage cells are known to be present in the brain parenchyma after a bone marrow transplant (see FIGS. 9b, c), but these donor-derived cells did not express GFP (FIG. 11), a further indication for the specificity of the L7-pcp-2 promoter. These results demonstrate that under physiological conditions, transplanted BMDCs not only fuse to pre-existing Purkinje neurons, but can also activate the Purkinje neuron-specific promoter, L7-pcp-2. Thus, in these cells the BMDC nucleus was reprogrammed after it fused to the Purkinje cell, enabling expression of the Purkinje-specific promoter L7-pcp-2. These results show that gene activation, only obtained previously in vitro in heterokaryons, can occur spontaneously in vivo. The results strongly suggest that the bone-marrow-derived nuclei are not only altered morphologically, but also reprogrammed in the adult Purkinje cell, as shown by the expression of the reporter gene GFP under the control of the L7-pcp-2 promoter.

These data show clearly that fusion is the underlying mechanism by which BMDCs contribute to Purkinje neurons. Fusion occurs spontaneously and physiologically to generate stable heterokaryons in the absence of selective pressure through genetic defects or drug treatment. The frequency increases over time, even though the blood-brain barrier opens only transiently. After a bone marrow transplant from a transgenic mouse ubiquitously expressing GFP, numerous GFP-positive cells were found to be binucleate Purkinje/BMDC heterokaryons in which the nuclei remained intact and distinct. Such heterokaryons increased in frequency with increasing age of the mouse. The morphology of the more than 300 GFP-positive cells analysed was typical of functional thriving Purkinje cells, with axons and full complex dendritic trees from which synaptic spines projected. Fusion of BMDC was specific to these cells, as no other neurons in this part of the brain expressed GFP after transplant. This finding is of particular interest, as Purkinje neurons are the most complex and elaborate in the cerebellum and have a critical function in balance and movement. Definitive proof that the binucleate cells resulted from fusion was obtained after transplantation of male bone marrow into female mice and detection of a Y chromosome in one of the two nuclei per heterokaryon.

To date, the only other example of BMDC fusion to tissue-specific cells in vivo is in the liver. Vassilopoulos, G., Wang, P. R. & Russell, D. W. Transplanted bone marrow regenerates liver by cell fusion. Nature 422, 901-904 (2003). Wang, X. et al. Cell fusion is the principal source of bone-marrow-derived hepatocytes. Nature 422, 897-901 (2003). The results of these studies are distinct from those reported here in several respects: first, although the initial frequency of fusion agrees with the data in this report (1/50,000), the hepatocyte/BMDC fusion product is not a stable heterokaryon. Instead, it proliferates extensively, resulting in millions of highly aneuploid progeny; second, the liver studies used strong selective pressure, the survival of a mouse with a lethal genetic disorder, tyrosinemia, as well as repeated drug administration. For survival, expansion of the rare BMDC/hepatocyte fusion events was absolutely necessary. The resulting karyotypic instability was presumably well tolerated because adult hepatocytes are typically multinuclear, polyploid and even aneuploid37-39. As 6% of donor bone-marrow-derived hepatocytes were diploid20, the possibility remains that a cell-fate change from BMDC to hepatocyte occurred before fusion, rather than after fusion with host hepatocytes. Thus, it is unclear which came first, a cell fate change or cell fusion.

In summary, the findings reported here are highly unexpected and significant for several reasons, including, for example, the following: heterokaryons formed spontaneously in vivo through the fusion of two disparate cell types, resulting in stably binucleate cells with equivalent chromosomal input. These data demonstrate that cell fusion in Purkinje neurons of the mouse brain can occur under physiological conditions without ongoing selective pressure. The result of this fusion is a heterokaryon containing a reprogrammed bone marrow nucleus, presumably through the increased dosage of regulatory proteins in the much larger Purkinje cell cytoplasm. Each GFP-positive fusion product, of the hundreds examined, was binucleate, and the frequency of this event increased with age.

Methods

Bone Marrow Transplantation.

Marrow was isolated under sterile conditions from 8-10-week-old C57BL/6 transgenic mice that ubiquitously expressed enhanced green fluorescent protein (GFP)42. Donor mice were killed by cervical dislocation, briefly immersed in 70% ethanol and their skin peeled back from a midline, circumferential, incision. After the femurs, tibias and humeri were removed, all muscle was scraped away with a razor blade and the bones were placed in 10 ml of calcium and magnesium-free, Hank's balanced salt solution (HBSS, Invitrogen, Carlsbad, Calif.) with 2.5% foetal calf serum (FCS; SH30072.03, HyClone, Logan, Utah) on ice for up to 90 min. The tips of the bones were removed and a 25-gauge needle containing 1 ml of ice-cold HBSS with 2.5% FCS was inserted into the marrow cavity and used to wash the marrow out into a sterile culture dish. Marrow fragments were dissociated by triturating through the 25-gauge needle, and the resulting suspension was filtered through sterile 70 μm nitex mesh (BD-Falcon, Franklin Lakes, N.J.). The filtrate was cooled on ice, spun for 5 min at 250 g, and the pellet was resuspended in ice-cold HBSS with 2.5% FCS to 8×10⁷ nucleated cells per ml. Simultaneously, 8-10-week-old C57BL/6 mice (Stanford) were lethally irradiated with two doses of 4.8 Gy 3 h apart. Each irradiated recipient received 125 μl of the unfractionated marrow cell suspension by tail-vein injection within 1 h of the second irradiation dose.

Harvesting of Brains.

Mice were sacrificed at various times after bone marrow transplantation. The mice received a lethal injection of pentobarbital (Sleepaway, Fort Dodge Animal Health, Fort Dodge, Iowa) and were immediately perfused with ice-cold phosphate buffer (PB) followed by 4% paraformaldehyde in PB. The brains were then removed and cryoprotected in a 20% sucrose/PB solution overnight. Thick tissue sections (35-50 μm) for antibody staining, enumeration of donor derived cell number and nuclear content were obtained on a sliding microtome (SM2000R; Leica, Bannockburn, Ill.). Thin sections for FISH were made on a cryostat (CM3050S; Leica) at 10-12 μm and mounted on gelatin-coated slides (Goldseal, Portsmouth, N.H.).

Antibody Staining.

Antibodies against GFP (mouse 1:1000; #A-11120; rabbit 1:2000; A-11122, Molecular Probes, Eugene, Oreg.), Calbindin (1:1000; C9848, Sigma, St Louis, Mo.), MAP2 (M2376; 1:100, Sigma), CD11b (1:100; #553308, BD Biosciences PharMingen, San Diego, Calif.), CD45 (1:200; #553076BD Biosciences PharMingen), F4/80 (#RM2900; 1:50, Caltag, Burlingame, Calif.), Iba 1 (1:1000, a gift from Y. Imai, National Institute of Neurosciences, Tokyo Japan), were applied for 12 h at 4° C. to the floating sections after pre-incubation in blocking solution for 2 h. When mouse or rabbit primary antibodies were used, anti-CD16/CD32 (1:200) was also included (#553142; BD Biosciences PharMingen). The sections were then incubated in appropriate secondary antibodies overnight at 4° C. The blocking solution contained 5% goat serum, 3% BSA and 0.3% Triton X-100.

FISH Analysis.

Thin sections (12 μm) of the cerebella from GFP-transplanted mice were processed for GFP using standard immunohistochemistry. The nuclei were then counterstained with To-Pro3. These sections were then viewed for the presence of GFP-positive Purkinje neurons and scanned at 1 μm optical section using a scanning confocal microscope (LSM510; Zeiss, Thornwood, N.Y.). The x- and y-position of the GFP-positive cell bodies were recorded with respect to the corners of the slide, to relocate the exact position after FISH. The FISH protocol was modified from ref. 43 and protocols from Applied Spectral Imaging (Carlsbad, Calif.). Briefly, sections were then dehydrated, treated with proteinase K at 45° C. for 7-15 min, rinsed in 2×SSC and denatured in 70% formamide in 2×SSC at 68° C. for 5 min. The slides were then dehydrated and warmed to 50° C. The X and Y chromosome probes were denatured and applied as directed (see CamBio and Applied Spectral Imaging website). After 36 h at 37° C., the probe was washed off in 2×SCC, before incubation in 2×SSC/0.1% NP40 at 50° C. for 2 min and mounted with Vysis DAPI mounting solution with 1:3000 To-Pro3.

Flow Cytometry and FACS Analysis.

Bone marrow was prepared as described above, with the exception that erythrocytes were lysed in lysis buffer (0.15 M NH4Cl, 1.0 mM KHCO3 and 0.1 mM Na2EDTA at pH 7.4) for 5 min on ice before incubation with propidium iodine (PI; final concentration 100 g ml-1) to exclude dead cells. Total unfractionated bone marrow (100 μl) from five L7/GFP-Pcp-2 transgenic, one GFP-transgenic and three wild-type mice, respectively, were used to acquire data to determine whether bone marrow cells (one million cells) expressed GFP, using a FACSCalibur (BD Biosciences, San Diego, Calif.). These experiments were repeated in triplicate. Data were analysed and presented with FlowJo v.4.3 software (Tree Star, Inc., Ashland, Calif.), displayed as a contour plot at 5% probability, as a function of side scattered versus GFP fluorescence. All animals were processed simultaneously.

All publications and patent applications mentioned in this specification are incorporated herein by reference to the same extent as if each individual publication or patent application were specifically and individually indicated to be incorporated by reference.

The above description is illustrative and not restrictive. Many variations will be apparent to those skilled in the art upon review of this disclosure. The scope of the invention should not be determined with reference to the above description, but instead should be determined with reference to the appended claims and the full scope of their equivalents.

We claim:

1. A method for treating a neuronal deficiency, comprising: administering bone marrow-derived cells to an individual having a neuronal deficiency, wherein the administering of the bone marrow-derived cells induces a formation of bone marrow-derived neurons in a nervous system of the subject; and ameliorating at least one symptom of the neuronal deficiency.

2. The method of claim 1, wherein said neuronal deficiency arises from a disorder selected from the group consisting of abnormalities of the central autonomic systems, congenital disorders and disorders arising from teratogen exposure, demyelinating diseases, diseases of peripheral nerves, disorders of the hypothalamus and pituitary, disorders of movement, disorders of the spinal cord and vertebral column, epilepsy, hypoxia, increased intracranial pressure, infectious disease, neoplasia, neurodegenerative disorders, neuronal disorders associated with aging and senile dementia, nutritional disorders, perinatal neuropathologies, radiation damage, schizophrenia, single gene disorders, toxic disorders, trauma, vascular disease, and psychiatric disorders other than schizophrenia.

3. The method of claim 2, wherein said neuronal deficiency is not a neuron deficiency arising from a disorder selected from the group consisting of: a lysosomal or peroxisomal disorder, Zellweger's disease, human immunodeficiency virus (HIV) infection, multiple sclerosis (MS), adrenoleucodystrophy, adrenomyeloneuropathy, a metachromatic leucodystrophy, a sulphatide lipidosis, globoid cell leucodystrophy, amyotrophic lateral sclerosis, amyotrophic lateral sclerosis with frontal lobe dementia, a bone marrow ablation treatment, lymphoreticular disorders, metastases of tumors which do not arise in the nervous system, infantile acid maltase deficiency (Pompe's disease), Ceroid lipofuscinosis, a deficiency of GM2 gangliosidase, Sanfilippo's disease, leucodystrophy, systemic lupus erythematosus, thrombophilia associated with antiphospholipid antibodies or polycythemia, and anemia including Sickle cell disease, beta-thalassemia major, and other thalassemias.

4. The method of claim 1, wherein said bone marrow-derived cells are autologous.

5. The method of claim 4, wherein said autologous bone marrow-derived cells are genetically modified.

6. The method of claim 1, wherein said bone marrow-derived cells are allogeneic.

7. The method of claim 6, wherein said allogeneic bone marrow-derived cells are genetically modified.

8. The method of claim 1, wherein said bone marrow-derived cells are administered in conjunction with a neuronal factor.

9. The method of claim 8, wherein said neuronal factor is selected from the group consisting of: nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene, insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF-1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-.beta. 1, -.beta.2, -.beta.3, -.beta.4, and -.beta.5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies).

10. The method of claim 8, wherein said neuronal factor is administered with the bone marrow-derived cells.

11. The method of claim 8, wherein said neuronal factor is administered separately from said bone marrow-derived cells.

12. The method of claim 11, wherein said neuronal factor is administered intrathecally.

13. The method of claim 1, further comprising the step of mildly damaging the nervous system of the individual.

14. The method of claim 1, wherein said bone marrow-derived cells are administered by vascular administration.

15. The method of claim 14, wherein said bone marrow-derived cells are administered by intravenous administration.

16. The method of claim 15, wherein said bone marrow-derived cells are administered by intravenous infusion.

17. The method of claim 16, wherein said intravenous infusion is into a peripheral vein.

18. The method of claim 1, wherein said bone marrow-derived cells are administered intrathecally.

19. The method of claim 1, wherein said bone marrow-derived cells are administered by direct administration into a site in said subject's nervous system.

20. The method of claim 19, wherein said site in the subject's nervous system is in the subject's central nervous system (CNS).

21. The method of claim 1, wherein said subject is a human.

22. A method for improving memory function in an individual with deficient memory function, comprising: administering bone marrow-derived cells to an individual having deficient memory function, wherein the administering of the bone marrow-derived cells induces a formation of bone marrow-derived neurons in a nervous system of the subject; and improving at least one memory function in said individual.

23. The method of claim 22, wherein said bone marrow-derived cells are autologous.

24. The method of claim 23, wherein said autologous bone marrow-derived cells are genetically modified.

25. The method of claim 22, wherein said bone marrow-derived cells are allogeneic.

26. The method of claim 25, wherein said allogeneic bone marrow-derived cells are genetically modified.

27. The method of claim 22, wherein said bone marrow-derived cells are administered in conjunction with a neuronal factor.

28. The method of claim 27, wherein said neuronal factor is selected from the group consisting of wherein said neuronal factor is selected from the group consisting of nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene, insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF-1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-.beta. 1, -.beta.2, -.beta.3, -.beta.4, and -.beta.5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies).

29. The method of claim 27, wherein said neuronal factor is administered with the bone marrow-derived cells.

30. The method of claim 27, wherein said neuronal factor is administered separately from said bone marrow-derived cells.

31. The method of claim 30, wherein said neuronal factor is administered intrathecally.

32. The method of claim 22, wherein said memory function is a short term memory function.

33. The method of claim 22, wherein improving said memory function comprises stabilizing said memory function.

34. A method for treating a neuronal deficiency, comprising: administering a bone marrow cell mobilization therapy to an individual having a neuron deficiency, wherein the administering of the bone marrow cell mobilization therapy induces formation of bone marrow-derived neurons in the nervous system of the subject; and ameliorating at least one symptom of the neuronal deficiency.

35. The method of claim 1, wherein the bone marrow derived neuron is a neuron derived by fusion of a bone marrow-derived cell with a neuron.

36. The method of claim 1, wherein the bone marrow-derived neuron is a heterokaryon comprising a nucleus derived from a bone marrow-derived cell and a nucleus from a neuron.

37. The method of claim 22, wherein the bone marrow derived neuron is a neuron derived by fusion of a bone marrow-derived cell with a neuron.

38. The method of claim 22, wherein the bone marrow-derived neuron is a heterokaryon comprising a nucleus derived from a bone marrow-derived cell and a nucleus from a neuron.

39. The method of claim 34, wherein the bone marrow derived neuron is a neuron derived by fusion of a bone marrow-derived cell with a neuron.

40. The method of claim 34, wherein the bone marrow-derived neuron is a heterokaryon comprising a nucleus derived from a bone marrow-derived cell and a nucleus from a neuron.
